(19) United States
(12) Patent Application Publication
(10) Pub. No.: US 2002/0016543 A1
(43) Pub. Date: Feb. 7, 2002

(54) Method for diagnosis of and prognosis for damaged tissue

(76) Inventor: Jenny A. Tyler, Cambridge (GB)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/828,070

(22) Filed: Apr. 5, 2001

Related U.S. Application Data
(60) Provisional application No. 60/195,629, filed on Apr. 6, 2000.

(51) Int. Cl. A61B 5/055

(52) U.S. Cl. 600/410; 600/411

(57) ABSTRACT

Tissue is analyzed by acquiring multiple sets of magnetic resonance signals from a stationary body part, organ or tissue, quantizing the signals pixel by pixel within an area of interest, determining at least one magnetic resonance parameter in each pixel, and correlating the determined magnetic resonance parameter with predetermined magnetic resonance parameters that have been correlated with selected biological parameters of the body part, organ or tissue. The magnetic resonance parameter can be longitudinal relaxation time, transverse relaxation time, magnetization transfer, or magnetization ratio. The determined quantized magnetic parameter may be coded by color or otherwise with regard to the selected biological parameter and spatially displayed.

### CROSS-REFERENCES TO RELATED APPLICATIONS

This patent application is related to and claims the benefit of provisional application No. 60/195,629 filed Apr. 6, 2000, for “Use of MRI as a Non-Invasive Outcome Measure of Cartilage Repair”.

### BACKGROUND OF THE INVENTION

This invention relates generally to the diagnosis of injured tissue and to evaluating the quality of repaired tissue, and more particularly the invention relates to the use of magnetic resonance values in that diagnosis and evaluation.

Magnetic resonance imaging (MRI) is a non-destructive method for the analysis of material. It is generally non-invasive and does not involve ionizing radiation. In very general terms, nuclear magnetic moments are excited using magnetic fields which rotate at specific frequencies proportional to the local static magnetic field. The radio frequency signals resulting from the precession of excited spins are received by using pickup coils. By manipulating the magnetic fields, an array of signals is provided representing different regions of the volume. These are combined to produce a volumetric image of the nuclear spin distribution of the body.

FIG. 62A is a prospective view, partially in section, illustrating coil apparatus in MR imaging system and FIGS. 62B-62D illustrate field gradients which can be produced in the apparatus of FIG. 62A. Briefly, the uniform static field B₀ is generated by the magnet comprising the coil pair 10. A gradient field G(x) is generated by a complex gradient coil set which can be wound on the cylinder 12. An RF field B₁ is generated by a saddle coil 14. A patient undergoing imaging would be positioned along the z axis within the saddle coil. In FIG. 62B an x gradient field is shown which is parallel to the static field B₀ and varies linearly with distance along the x axis but does not vary with distance along the y or z axes. FIGS. 62C and 62D are similar representations of the y gradient and z gradient fields, respectively.

FIG. 63 is a functional block diagram of convention imaging apparatus. A computer 20 is programmed to control the operation of the MRI apparatus and process free induction decay (FID) signals detected therefrom. The gradient field is energized by a gradient amplifier 22 and the RF coils for impressing an RF magnetic moment at the Larmor frequency is controlled by the transmitter 24 and the RF coils 26. After the selected nuclei have been flipped, the RF coils 26 are employed to detect the FID signal which is passed through the receiver 28 and then through digitizer 30 for processing by computer 20.

MRI has heretofore been used in the study of the human body, particularly in imaging blood flow, organs of the body and abnormal tissue therein, and in studying neurological impairments that are not associated with structural abnormalities by imaging the brain. The use of MRI images in these studies requires that the differences in tissue can be readily imaged and necessarily leads to subjective evaluation.

### BRIEF SUMMARY OF THE INVENTION

In accordance with the invention, magnetic resonance parameters are used in the diagnosis of and prognosis for damaged tissue. More particularly, magnetic resonance parameters are quantized for a body part, organ or tissue sample in an area of interest, on a pixel-by-pixel basis. The quantized parameter values of the sample are correlated to quantized parameter values previously determined for healthy tissue structures and for damaged tissue structures and for types of repair tissue.

In a particular application, the invention is directed to the assessment of cartilage damage and cartilage repair. For pixels of a predetermined size, MRI parameters are quantized in areas of interest. These qMRI values are correlated to previously determined parameter values for healthy tissue structures and for damaged tissue structures, and for types of repair tissue. Additionally, images can be formed based on the qMRI parameter values.

In specific embodiments, the MRI parameters can be relaxation time (T₁ or T₂), magnetization transfer (MT), or magnetization ratio. Known MRI data acquisition techniques are employed to collect the signal data on a pixel-by-pixel basis for use in calculating the MRI parameter values. Pixel size is preferably selected to gain a satisfactory signal-to-noise ratio at the expense of a lower resolution. Healthy tissue structures and damaged tissue structures and types of repair tissue are determined utilizing biomedical techniques, such as histology, biochemistry, electron microscopy, histochemistry and others. The range of values for each Magnetic Resonance parameter for each of healthy tissues and damaged tissues and types of repair tissue can be color coded to provide a spatial map of pixels to provide a spatial picture of the quality of tissue repair.

The invention and objects and features thereof will be more readily apparent from the following detailed description and appended claims when taken with the drawing.

### BRIEF DESCRIPTION OF THE DRAWINGS

FIG. 1 illustrates MRI scans from an osteochondral plug demonstrating the sequence of acquisition for qMRI analysis.

FIG. 2 illustrates a definition of masks 2, 5 and 6 for qMRI analysis of cartilage and bone.

FIG. 3 illustrates automated output of histograms for C4 mask 3.

FIG. 4 illustrates output of histograms for C4 mask 4.

FIG. 5 illustrates automated output of histograms for C4 mask 8.

FIG. 6 illustrates C128 (ACD1+HAED, vital flap) zero time point histology showing detail of masks 2, 3, 6 and 7,

FIG. 7 illustrates C128 (ACD1) HAED,vital flap) zero time point definition of masks 2, 3, 6 and 7 for qMRI analysis.

FIG. 8 illustrates automated output of histograms for C128 mask 2.

FIG. 9 illustrates automated output of histograms for C128 mask 3.

FIG. 10 illustrates automated output of histograms for C128 mask 6.

FIG. 11 illustrates output of histograms for C128 mask 7.

FIG. 12 illustrates C129 (ACD1+HAED,vital flap) 8 weeks histology and MRI showing detail of masks 2, 3, 4 and 5.

FIG. 13 illustrates automated output of histograms for C129 mask 2.

FIG. 14 illustrates automated output of histograms for C129 mask 5.

FIG. 15 illustrates automated output of histograms for C129 mask 4.

FIG. 16 illustrates automated output of histograms for C129 mask 3.

FIG. 17 illustrates graphs comparing mean values of T2, MT, T1 and ADC for ACD1 defects C128 and C129 with the unoperated control, C4.

FIG. 18 illustrates C146 (ACD1+HAED,vital flap) 8 weeks histology and MRI showing detail of masks 2, 3 and 4.

FIG. 19 illustrates automated output of histograms for C146 mask 2.

FIG. 20 illustrates automated output of histograms for C146 mask 3.

FIG. 21 illustrates C131 (ACD1+HAED, partially devitalized flap) 6 weeks histology and MRI showing detail of masks 2 and 3.

FIG. 22 illustrates automated output of histograms for C131 mask 2.

FIG. 23 illustrates output of histograms for C131 mask 3.

FIG. 24 illustrates C137 (virtual ACD1+PEG Triacrylate, partially devitalized flap) 10 weeks histology and MRI showing detail of masks 2 and 3.

FIG. 25 illustrates automated output of histograms for C137 mask 2.

FIG. 26 illustrates automated output of histograms for C137 mask 3.

FIG. 27 illustrates automated output of histograms for C137 mask 4.

FIG. 28 illustrates automated output of histograms for C137 mask 4a.

FIG. 29 illustrates automated output of histograms for C137 mask 5.

FIG. 30 illustrates selected MR scans for qMRI analysis of spontaneously healed ACD3 defects C1, C2 at 8 weeks and C78, C79 at 6 months.

FIG. 31 illustrates histology and MRI of spontaneously healed ACD3 defects C78 and C79 at 6 months.

FIG. 32 illustrates histology and MRI showing detail of masks 2, 4, 3, and 7 for C78 and C79.

FIG. 33 illustrates automated output of histograms for C78 mask 2.

FIG. 34 illustrates automated output of histograms for C78 mask 4.

FIG. 35 illustrates automated output of histograms for C79 mask 3.

FIG. 36 illustrates automated output of histograms for C79 mask 7.

FIG. 37 illustrates histology and MRI of spontaneously healed ACD3 defects C1 and C2 at 8 weeks.

FIG. 38 illustrates automated output of histograms for C2 mask 3.

FIG. 39 illustrates automated output of histograms for C2 mask 4.

FIG. 40 illustrates automated output of histograms for C2 mask 5.

FIG. 41 illustrates automated output of histograms for C1 mask 6.

FIG. 42 illustrates automated output of histograms for C1 mask 7.

FIG. 43 illustrates histology and MRI for C77 (ACD3+HAED) at 8 weeks.

FIG. 44 illustrates automated output of histograms for C77 mask 2.

FIG. 45 illustrates automated output of histograms for C77 mask 3.

FIG. 46 illustrates automated output of histograms for C77 mask 8.

FIG. 47 illustrates histology and MRI for C76 (ACD3+HAED) at 8 weeks.

FIG. 48 illustrates automated output of histograms for C76 mask 6.

FIG. 49 illustrates automated output of histograms for C76 mask 5.

FIG. 50 illustrates graphs comparing mean values of T2, MT, T1 and ADC for ACD3 defects C1, C2, C78, C79, C77, and C76.

FIG. 51a-c illustrates adjacent slices (150 μm) from a 3D data set showing views from one side of defect C137 (virtual ACD1+PEG Triacrylate) to the other.

FIG. 52 illustrates scoring ACD1 defects from 3-D MRI scans. Degree of filling in the defect.

FIG. 53 illustrates scoring ACD1 defects from 3-D MRI scans. Integration of the repair tissue.

FIG. 54 illustrates scoring ACD1 defects from 3-D MRI scans. Smoothness of surface repair.

FIG. 55 illustrates scoring ACD1 defects from 3-D MRI scans. Disruption of trabecular and subchondral bone.

FIG. 56 is a table summarizing scores of ACD1 defects from 3D MRI scans.

FIG. 57 illustrates slices from a 3D data set of C181 showing views and scoring of an OCT graft in the medial condyle.

FIG. 58 illustrates slices from a 3D data set of C182 showing views and scoring of an OCT graphs in the lateral condyle.

FIG. 59 illustrates slices from a 3D data set of C174 showing views and scoring of an OCT graft in the lateral condyle.

FIG. 60 illustrates slices from a 3D data set of C173 showing views and scoring of an OCT graft in the medial condyle.

FIG. 61 is a table summarizing scores OCT grafts from 3-D MRI scans.

FIGS. 62A-62D illustrate the arrangement of conventional MRI apparatus and magnetic fields generated therein.

FIG. 63 is a functional block diagram of MRI apparatus.

FIGS. 64A-64C illustrate MRI values as spatial information.

FIG. 65 illustrates MRI color values in FIGS. 64A-64C.

### DETAILED DESCRIPTION OF ILLUSTRATIVE EMBODIMENTS

The analysis of tissue based on quantized MRI data and magnetic resonance values of tissue on a pixel by pixel basis has been demonstrated using tissue samples with articular cartilage damage (ACD). ACD-1 designates an articular cartilage defect limited to the cartilage compartment. ACD-2 designates a defect that exceeds the depth of the cartilage compartment and enters into but does not penetrate the subchondral bony plate. ACD-3 designates an articular cartilage defect that penetrates through the subchondral plate into the trabecular bone marrow. Use of MRI as an outcome measure has been demonstrated in monitoring the induction of effective cartilage repair with a Bone Morphogeneric Protein-2 (BMP-2) device.

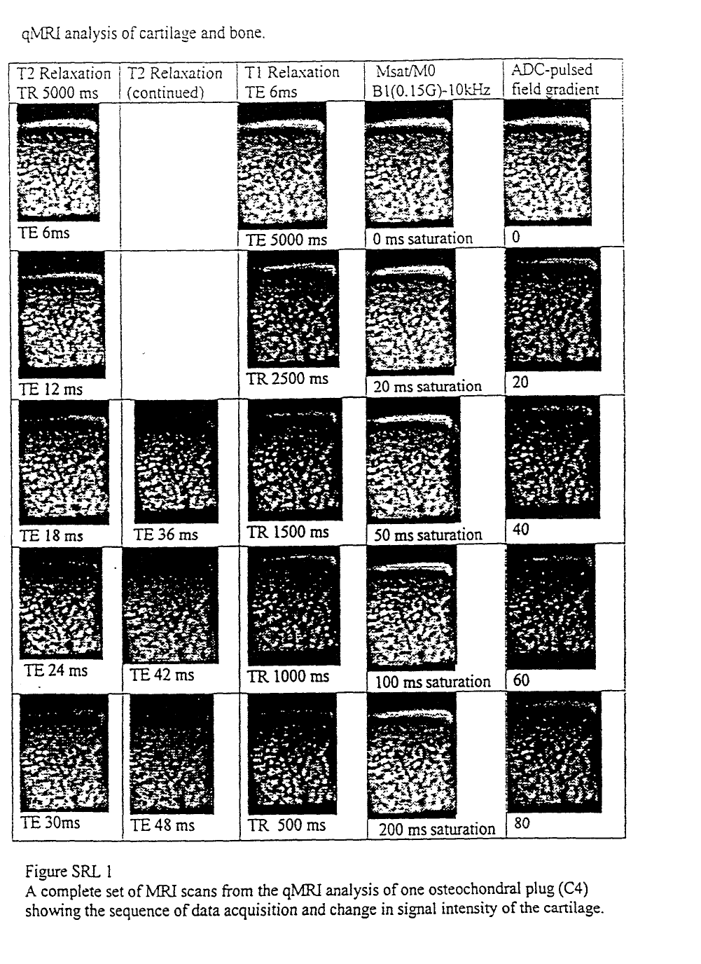

Repair was studied in four types of model (Table 1) at 3, 8 and 24 weeks; ACD1 and ACD3 defects in the trochlea implanted with a BMP-2 matrix, spontaneous repair in ACD3 defects and osteochondral plugs transplanted as autografts into ACD3 defects in the condyle. For the present study, a 3D data set (12 min) and a complete set of 23 qMRI scans (140 min) as shown in FIG. 1 were acquired for each sample.

TABLE 1
Summary of MRI acquisition.

| | | | Adjacent | |
|---|---|---|---|---|
| | qMRI sequence | Repair Tissue | Cartilage | |
| Sample | (23 images) | masks | masks | 3D data sets |
| ACD3, | 28 | 112 | 56 | 28 |
| spontaneous | | | | |
| ACD3 + | 69 | 138 | 138 | 65 |
| matrix | | | | |
| ACD3 + | 12 | 24 | 24 | 12 |
| grafts | | | | |
| ACD1 + | 32 | 96 | 64 | 28 |
| matrix | | | | |
| Unoperated | 16 | 16 | — | 16 |
| cartilage | | | | |
| TOTAL | 157 | 386 | 282 | 149 |

After MRI, the specimens were preserved in formalin for histological processing. The qMRI data output is automated and any region of interest can be chosen for analysis by outlining a mask on any of the 23 images with a computer-aided tool. The purpose of these experiments was to compare the MR parameters for different types of repair tissue with hyaline cartilage and a subjective decision as to where to draw the masks was taken with reference to changes in MR contrast (see Figure SRL 30), and the histology. qMRI images displayed in the Figures indicate which regions were chosen for analysis.

All defects to study matrix-induced repair were created in the trochlea. Grafts were transplanted into defects in the condyle. Samples referred to as ACD1 defects+matrix contained a composite material defined by one of six matrices mixed with minced synovium and covered by a sutured vital or partially devitalized (frozen/thawed once) synovial flap. The final concentration of growth factor was 250 μg/ml BMP-2 and 40 ng/ml IGF-1. ACD3 defects+matrix contained the same matrix with the same concentration of growth factor but no mince or flap. Twenty-eight ACD3 defects contained no matrix but were left to repair spontaneously. Eight ACD1 defects also contained no matrix, but were not examined by MRI as the defects remained completely empty. Four ACD3 condylar grafts contained two (5 mm diam×8 mm deep) osteochondral plugs transplanted from the trochlea and eight contained single autografts.

Magnetic Resonance Imaging

All protons within living tissues have an inherent magnetic moment and spin randomly giving rise to no net magnetization or direction. When a knee or biopsy specimen is placed within the magnetic field of the MR scanner, the protons continue to spin but align themselves parallel or antiparallel to the direction of the field (B₀ ) corresponding to low and high energy states respectively. In the course of an MR examination, a radiofrequency (RF) pulse (B₁) is applied to the sample from a transmitter coil orientated perpendicular to B₀ and the protons are momentarily tilted out of alignment; the precession of the induced net transverse magnetization around the axis of the static B₀ field produces a voltage across the ends of the receiver coil which is detected as the MR signal.

Any variation in the environment of water protons within cartilage or repair tissue, due to compression or changes in matrix concentration, hydration, or amounts of interfibrillar water within the collagen fibrils will lead to altered rates of relaxation of the induced MR signal. The MR parameters that give rise to this altered contrast can be expressed as a quantitative value (qMRI).

Spatial Resolution

The digital resolution in a two dimensional MR image is determined by the slice thickness, typically 1-4 mm and the pixel size, typically 100-1000 μm. The pixel size is pre-determined at the start of the experiment by choosing the field of view (FOV) for the object of interest and the size of the matrix, say 128, 256 or 512. It is now straightforward to measure cartilage dimensions of thickness or volume from an MR image. In such cases, or where images are to be scored visually, the highest possible resolution that provides enough signal in the allocated scan time is recommended. However, where quantitative values are to be acquired, it may be appropriate to gain increased signal to noise per pixel at the expense of a lower resolution image.

Measurement of T1 and T2 Relaxation

As a result of random thermal motion, the proton spins within a sample lose coherence with one another and the signal decays. The time taken for the MR signal to return to zero depends on many factors, one is the rate at which the energized spins lose their excess energy to their immediate environment, called spin-lattice or T1 relaxation which affects mainly magnetization parallel to B₀ and leads to a net loss of energy from the spin system.

Another is the slight difference in frequency in the spins of neighboring protons which tend to drift out of alignment with one another losing their phase coherence and this is called the spin-spin or T₂ relaxation. This therefore affects the transverse component of the magnetization but does not cause a net loss of energy.

There are alternative ways of measuring T1 and T2 relaxation times. In the present experiments, the T2 component of signal decay was assessed by keeping the repetition time (TR; the time interval between one RF pulse and the next) constant and varying the echo time (TE; the time interval between the RF pulse and sampling the MR signal). A representative example of an experiment to measure T2 relaxation rates in cartilage is shown in Figure SRL1. Each image is of the same sample scanned at a repetition time (TR) of 1500 ms but with increasing echo times (TE) of 6,12,18,24,30,36,42 and 48 ms; total imaging time 12 minutes. Less signal, is apparent at the longer echo times because it has decayed before it was recorded. The mean T₂ value of cartilage within mask 5 of specimen C4 was automatically calculated to be 13.8±0.5 ms from the decay curve based on a single exponential.

In a similar manner, sequential images of the same sample were acquired and processed to calculate a T1 relaxation time of 907±76 ms but in this case the TE was kept constant at 6 ms and the TR was decreased from 5000 to 500 ms.

Magnetisation Transfer (MT)

Protons within the joint are either freely mobile or bound to relatively immobile polymers. In a magnetization transfer experiment, data from a normal spin echo sequence is acquired and then the sample is re-imaged using a weak (0.15 G) source of radiofrequency energy 10 kHz off resonance from the frequency of freely mobile water; the signal of water in contact with macromolecules is selectively saturated and suppressed. As energy is transferred from macromolecules to free water, signal is lost until eventually an equilibrium is reached which is characteristic of that tissue. Any colloidal system with a polymeric structure and freely exchangeable protons will display MT suppression and the degree of MT saturation achieved is proportional to the concentration of the polymer, it's affinity with water and the degree of crosslinking. Normal healthy cartilage shows a marked MT suppression, typically more than 80%, following an MT sequence compared to a spin echo image with no MT (FIG. 1, 0-200 ms saturation). The mean value for the cartilage within mask 5 of specimen C4 was calculated to be 87.5% or 0.125 as the Msat/M0 ratio of the signal with and without MT. This effect is thought to be due in part to the crosslinked collagen network, which raises the possibility as to whether such measurements could be used to monitor the formation of a new Type II collagen network during repair.

Diffusion

The diffusion co-efficient was estimated by acquiring 5 images with a pulsed field gradient set at 0, 20, 40, 60 and 80. Total water content (M0) was computed from the same data. It should be noted that the measurements of diffusion co-efficient by this method were not consistent even with phantom samples and should therefore be considered unreliable. All other MR parameters were the same for bulk measurements or slice selected images and could be acquired in a consistent manner.

Automated Report Generation

Following acquisition of the qMRI sequence (23 images), one of the images was displayed on the screen and a region of interest or mask outlined with a computer-aided tool. The T1, T1 sat, T2 and MT ratio for each pixel within that delineated region was automatically calculated based on a single exponential decay and printed. For one embodiment we chose to print out a histogram plot of the frequency distribution of each parameter. As the distribution is approximately Gaussian, the mean value and standard deviation of the total number of pixels defined by the mask was also given to provide a working MR definition of that region of the specimen. The quantitative information is acquired individually for every pixel within the field of view. It is therefore possible to calculate mean MR values in the same way for muscle, fat or any other tissue of interest and compare them with the changes occurring within cartilage during the repair process.

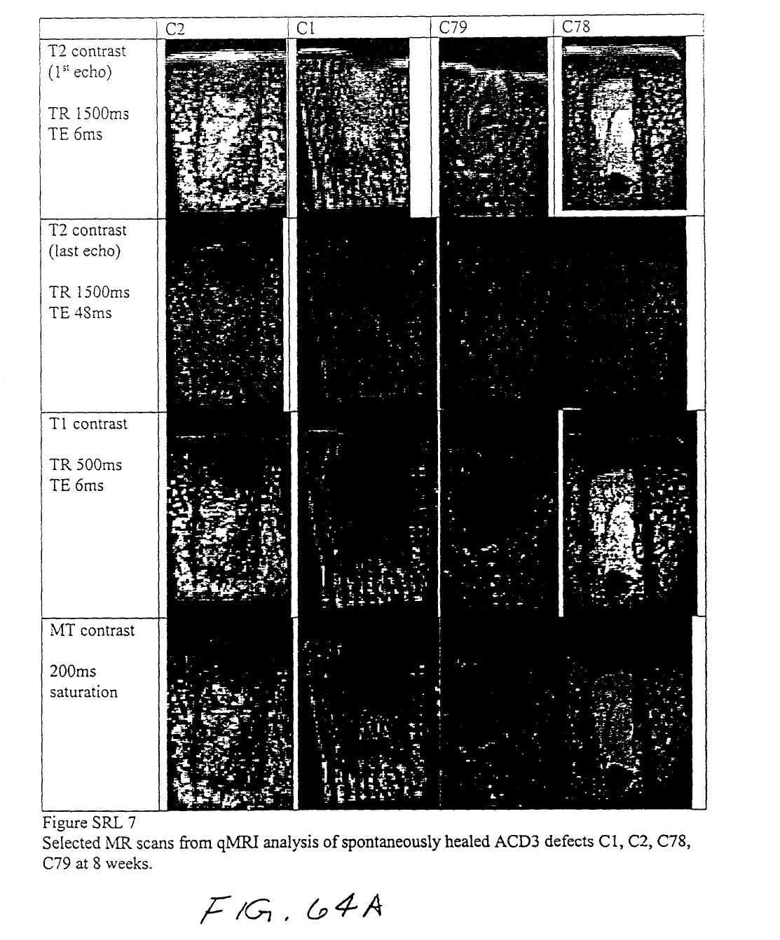

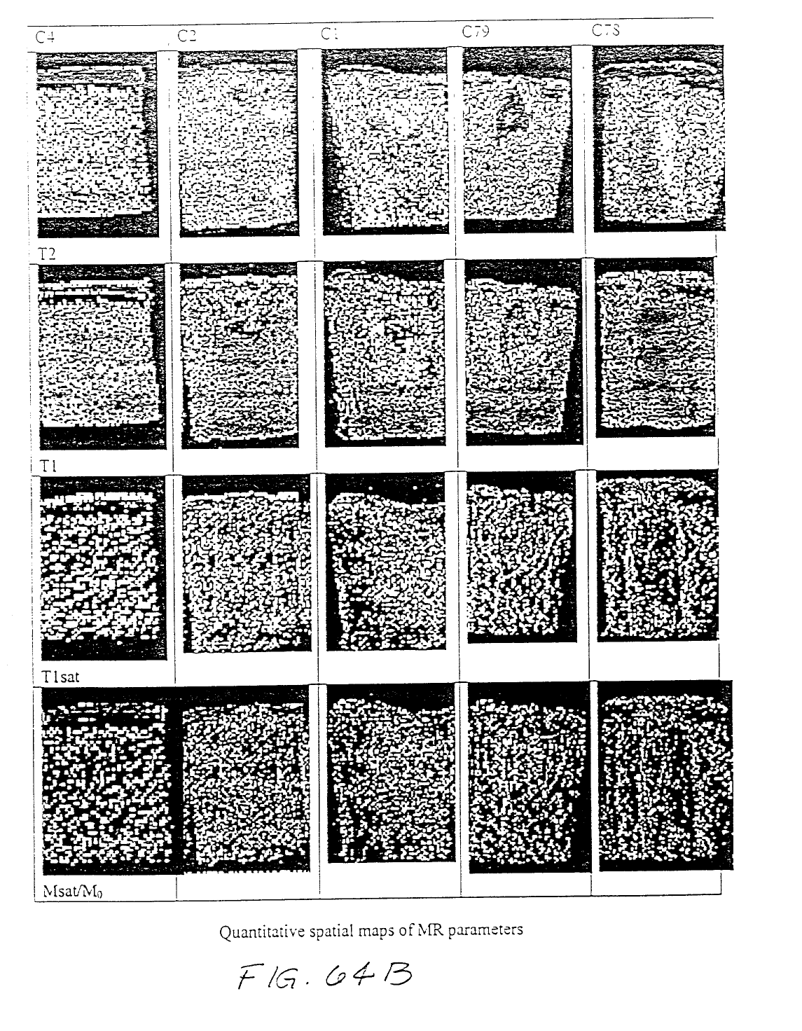

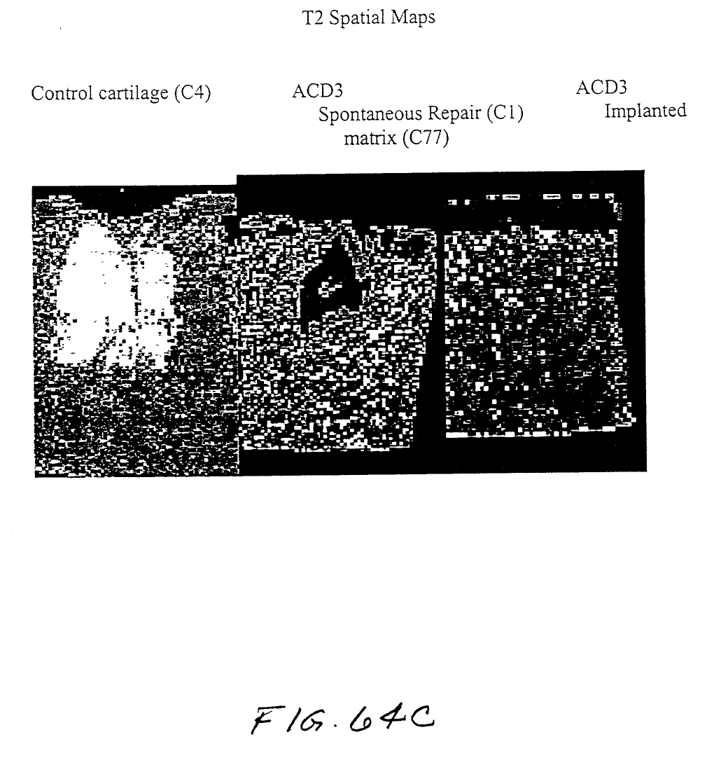

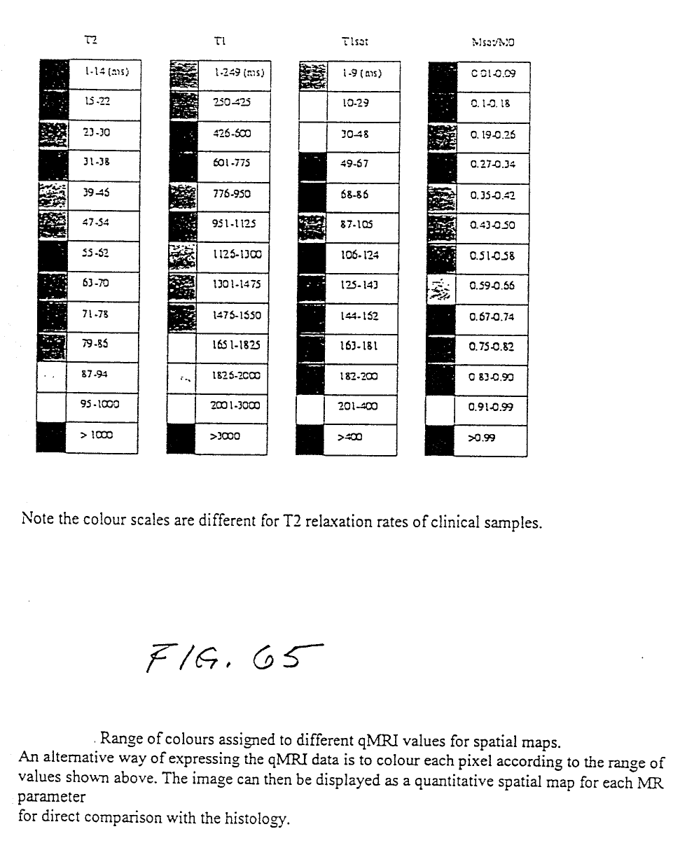

Prints outs of each distribution map of T2, T1, MT values or total water content are generated pixel by pixel to provide spatial information of where changes occur, as illustrated in FIGS. 64A-64C. FIG. 65 illustrates MRI color values for T2, T1, T1sat, Msat/M₀.

Control Samples

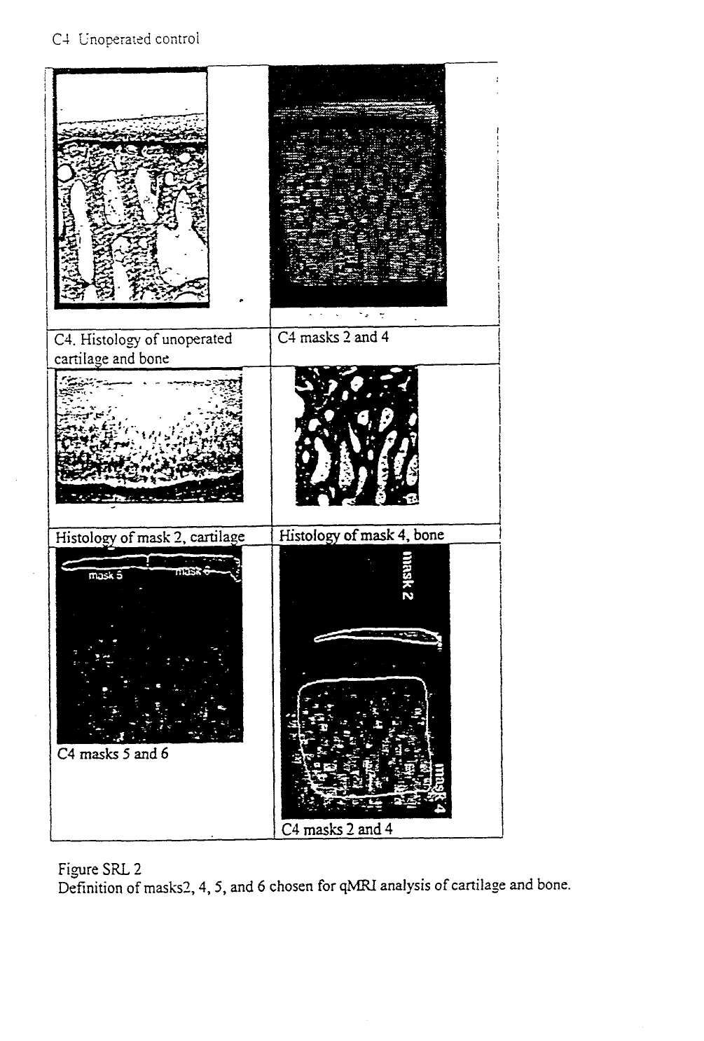

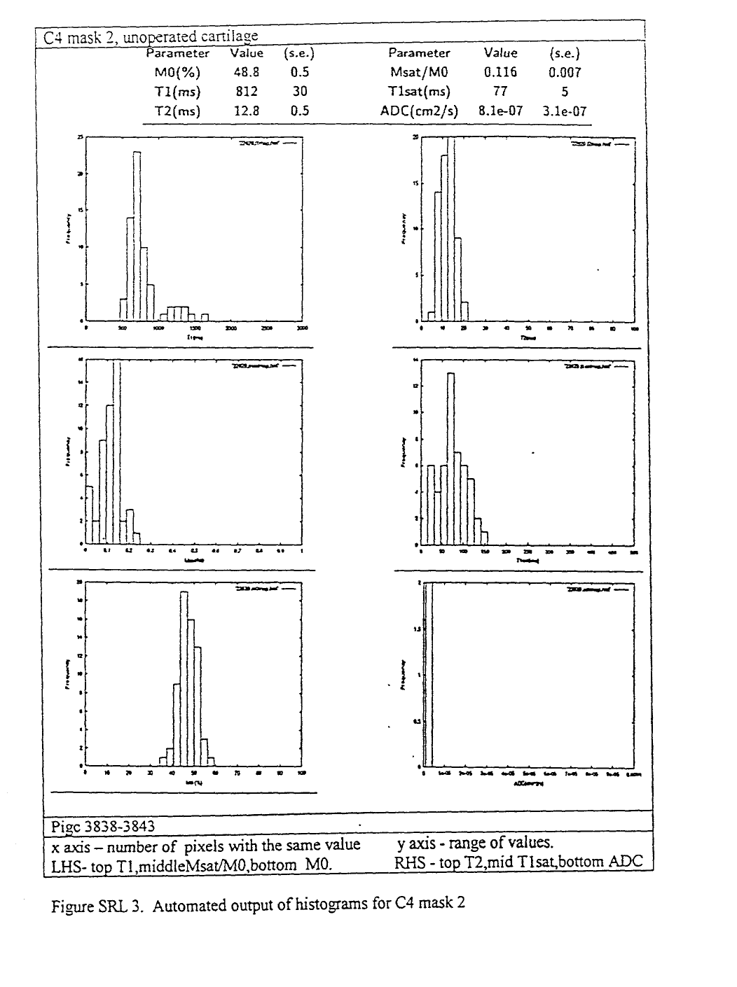

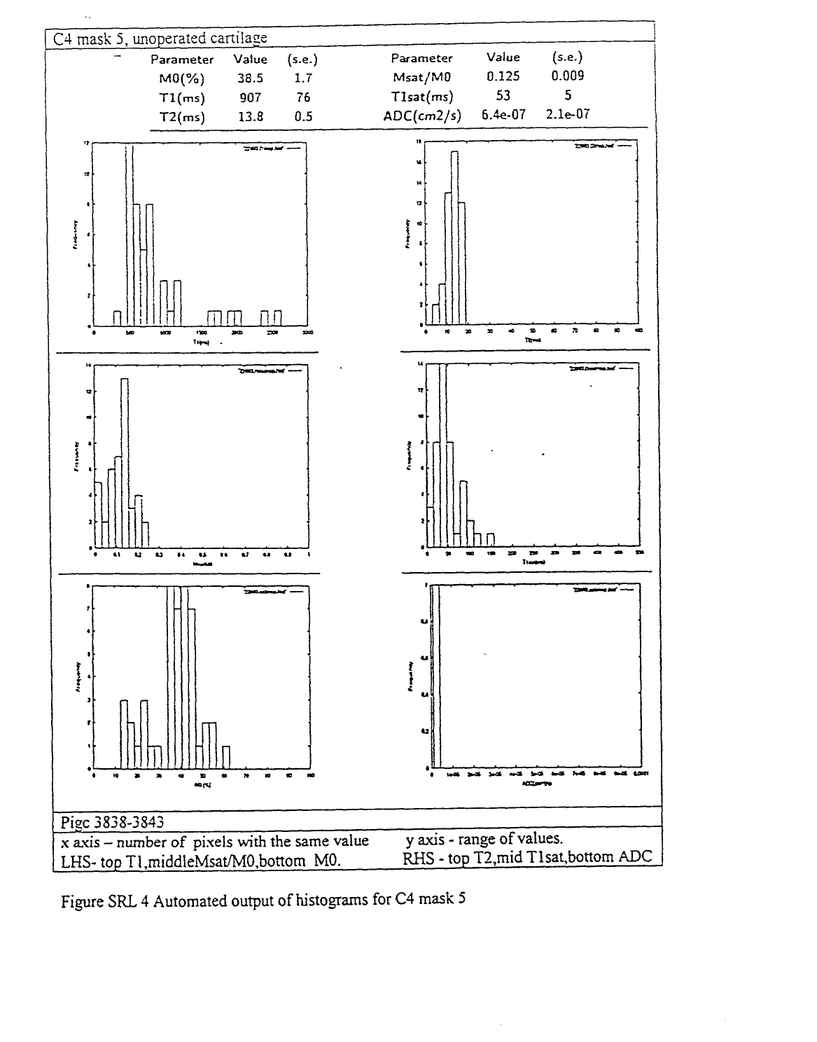

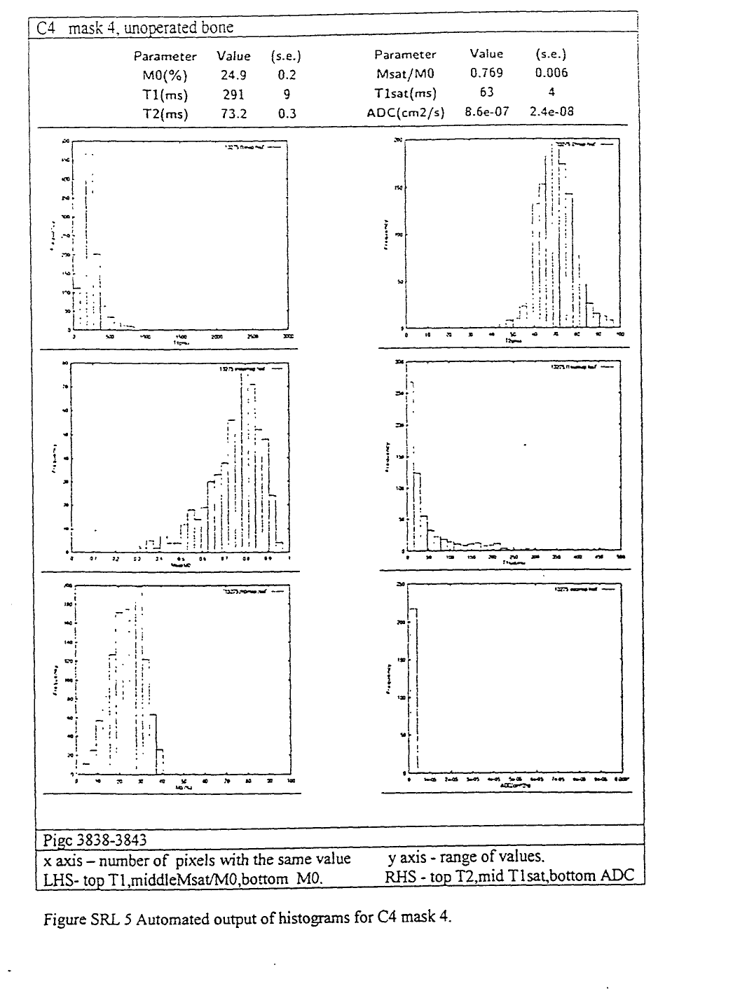

Osteochondral plugs for use as control cartilage were excised from 16 left knee joints that had received no surgical intervention. However, it is possible they may have been subjected to a slight increase in load as the right knee was bandaged with a clinical restraint. The detail of regions chosen for analysis of cartilage and bone in one control sample, C4 are shown in FIG. 2 together with a picture of the histology equivalent to those masks. Automated print outs of mean MR values for cartilage and bone calculated from masks 5,2 and 4 are shown in FIGS. 3-5. The plots for all histograms in the Report show T1 and T2 (top left and right) Msat/M0 and T1sat (middle left and right) and Mo (%) and ADC cm2 /sec (bottom left and right). It can be clearly seen that both the mean and spread of particularly the T2 values and Msat/M0 ratio are significantly different in bone compared to cartilage.

Samples with Implanted Defects

MR data was obtained in a similar manner for samples with implanted defects, and values for regions of ACD1 or ACD3 repair tissue and cartilage adjacent to either side of the defect were compared to the unoperated cartilage controls and other joint tissues.

ACD1-HAED Matrix

C128, Zero Time

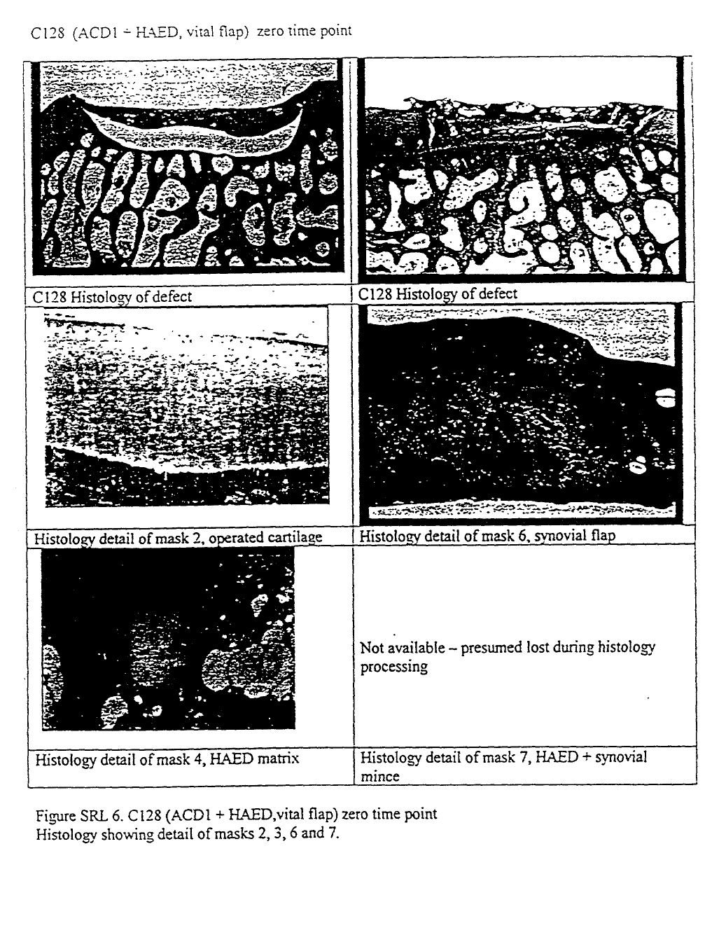

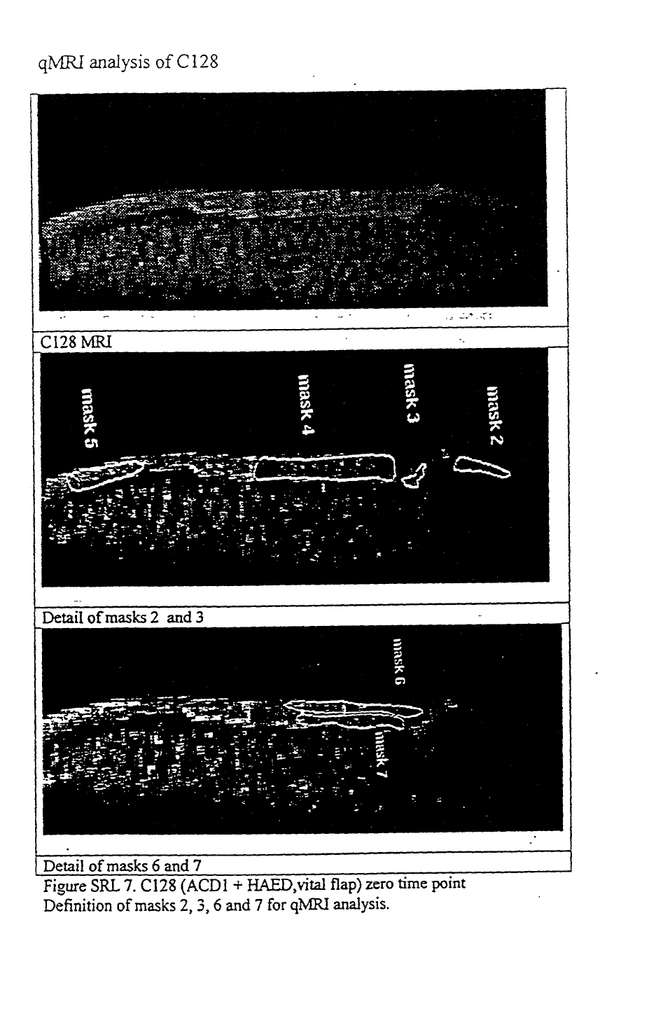

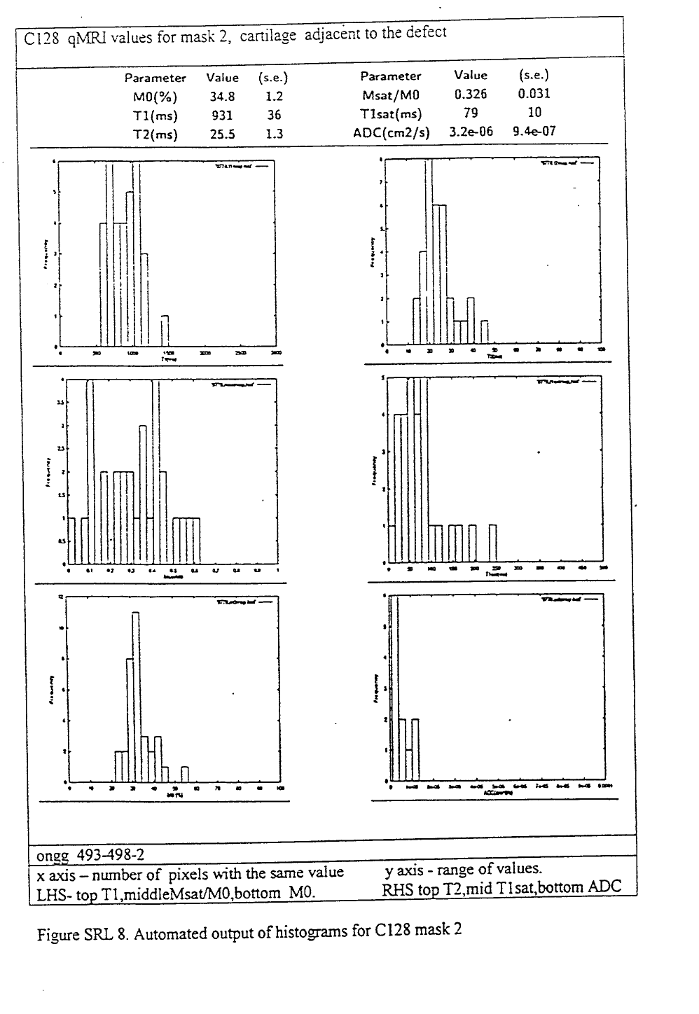

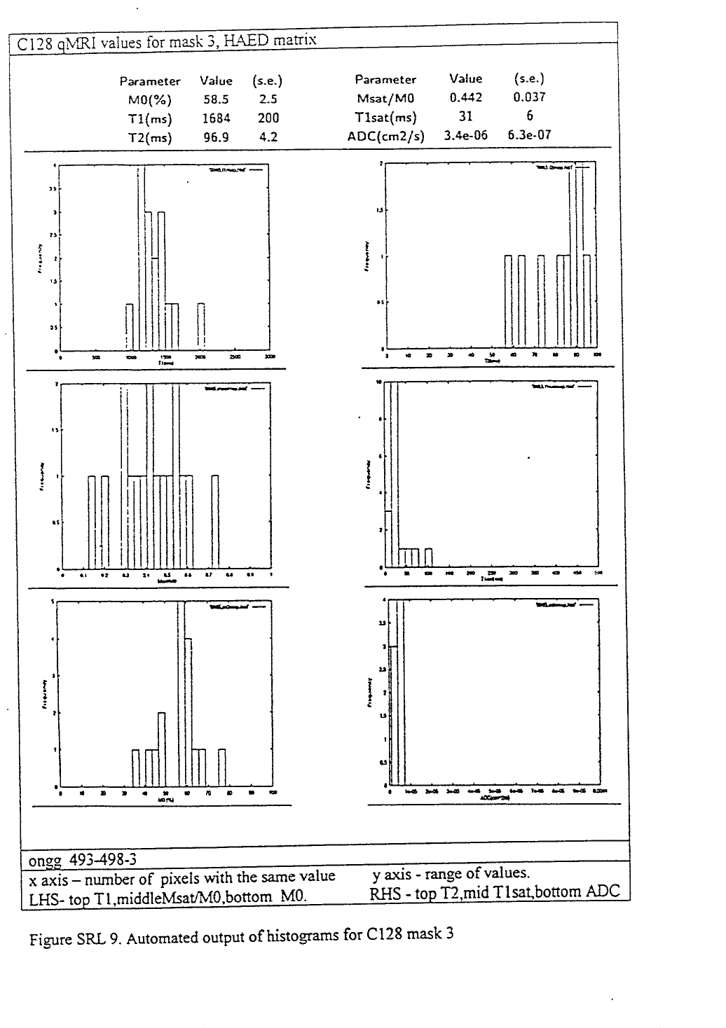

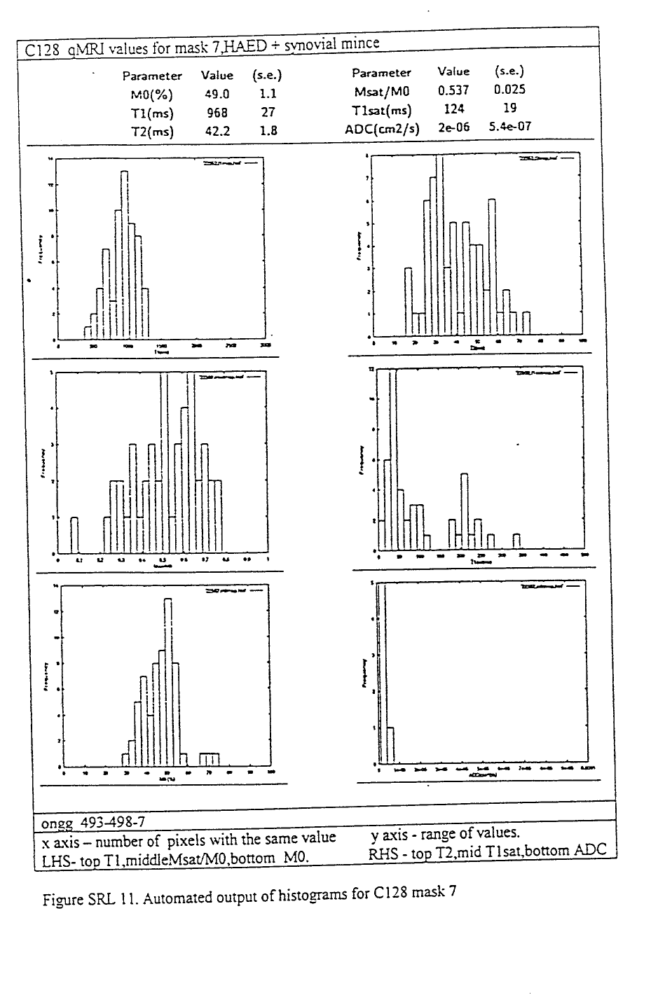

FIGS. 6-11 are views of C128; an ACD1 defect filled with HAED and covered with a vital synovial flap that was examined immediately after implantation. This confirms that the composite mixture of the original matrix at the time of implantation has very different MR characteristics to hyaline cartilage. The synovial flap, which is composed of fibrous connective tissue also has very different MR characteristics to hyaline cartilage.

C129, Good Repair at 8 Weeks

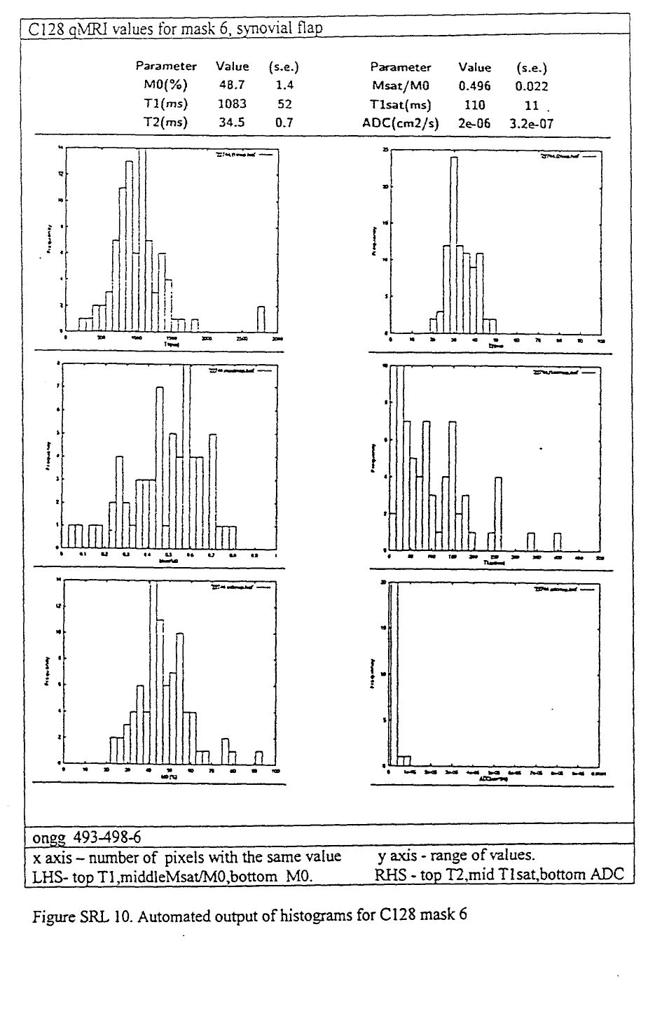

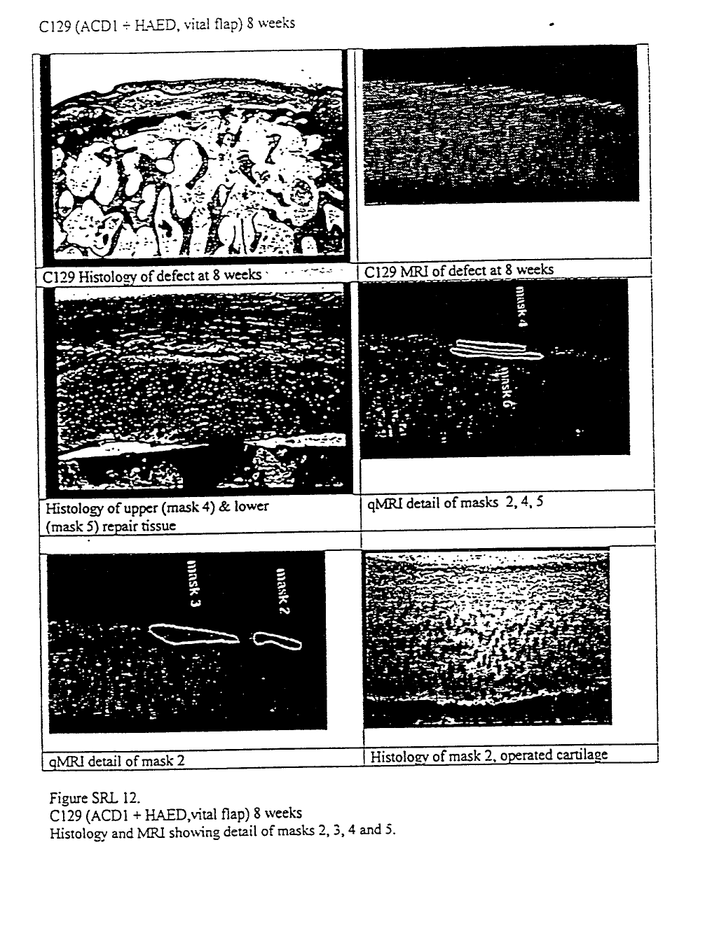

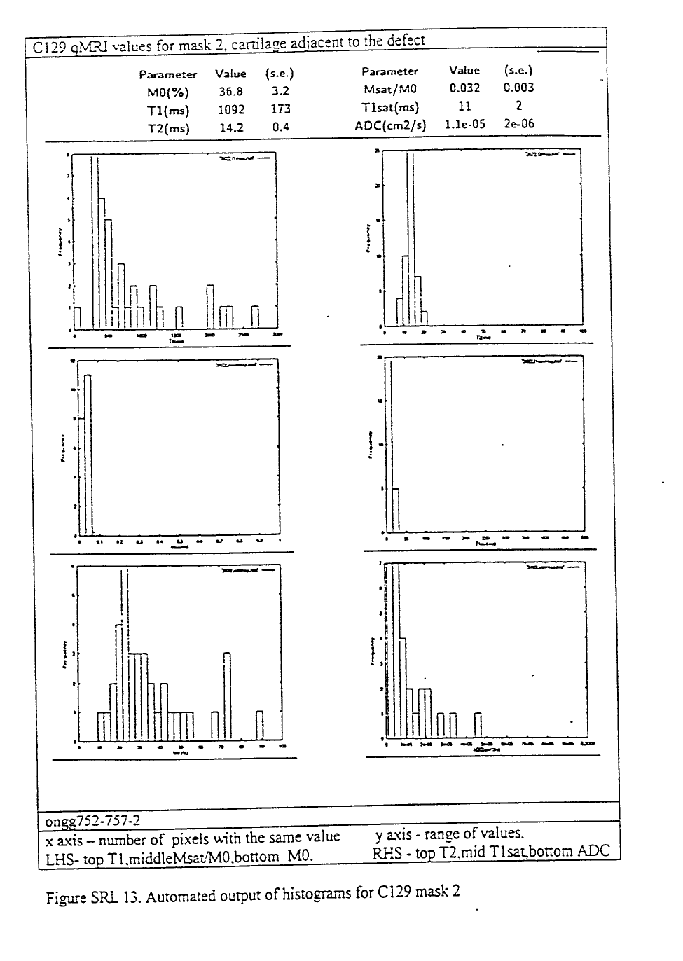

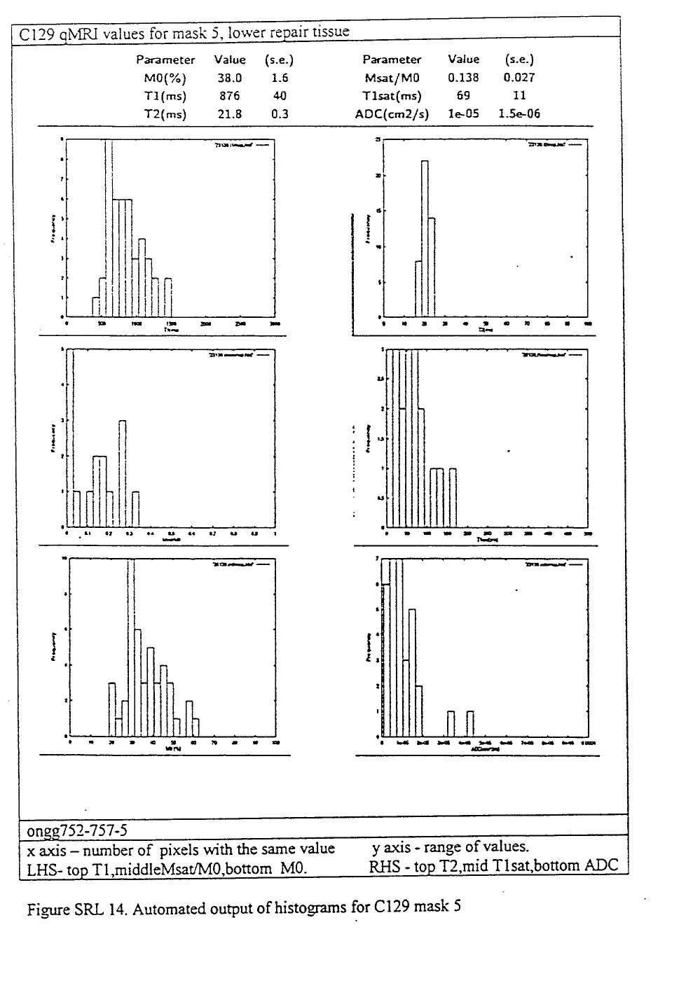

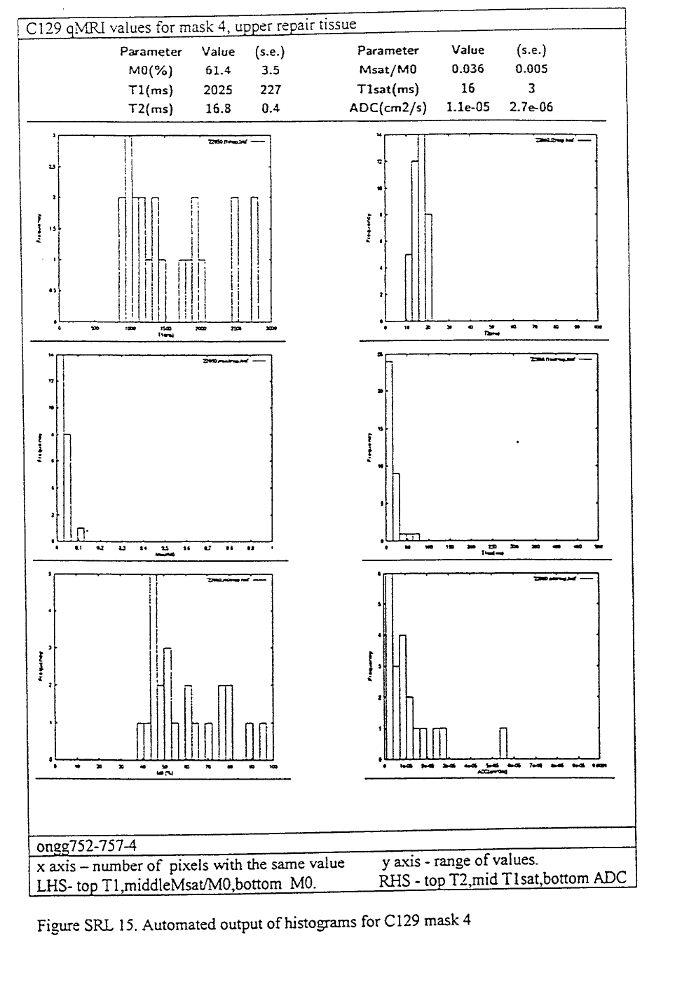

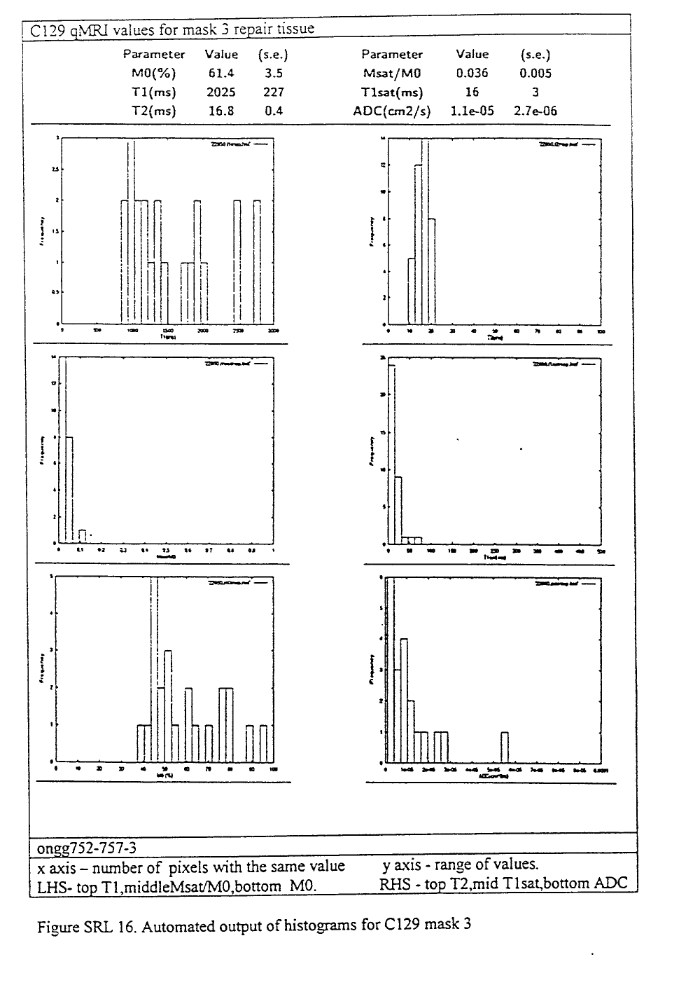

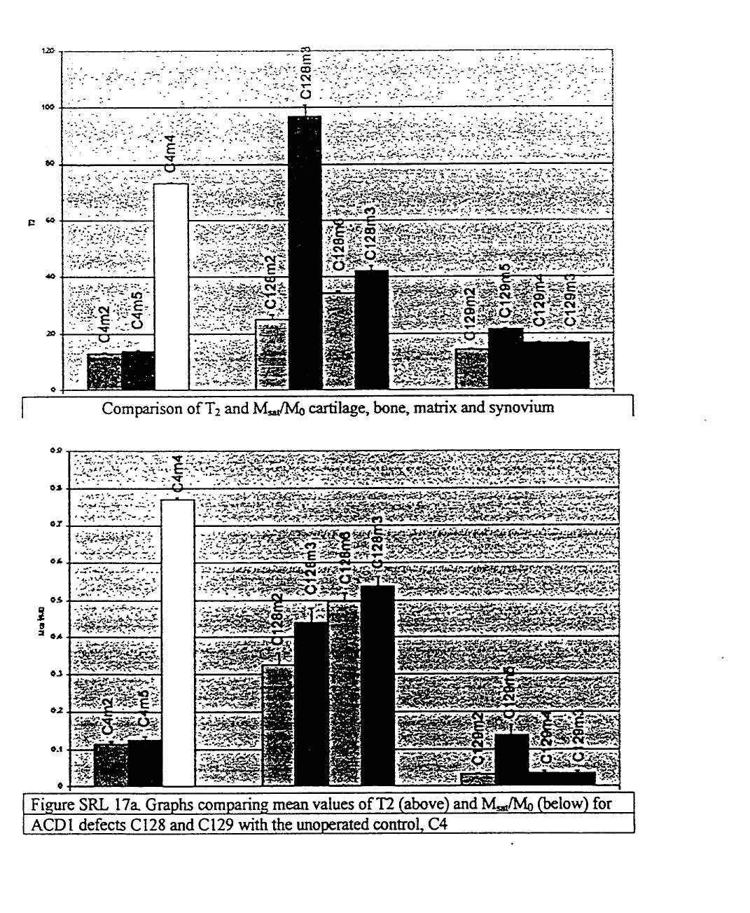

FIGS. 10-16 are views of C129; a second ACD1 defect filled with HAED and covered with a vital synovial flap that was examined 8 weeks after implantation. The histology shows complete chondrogenic transformation of the lower repair tissue and the implanted material now has very different MR characteristics. A graph summarizing those changes is shown in FIGS. 17a and b. The T2 relaxation rate is similar to normal cartilage. The MT, magnetization transfer (Msat/M0 ratio) also indicates a high concentration of polymer and/or a high degree of crosslinking. The T1 relaxation rate for the lower but not the upper repair tissue is within the cartilage range. The diffusion co-efficient (ADC) for the repair tissue and cartilage adjacent to the defect is unusually high in this sample but the reason for this is not known.

C146 and C131, Partially Filled at 8 Weeks

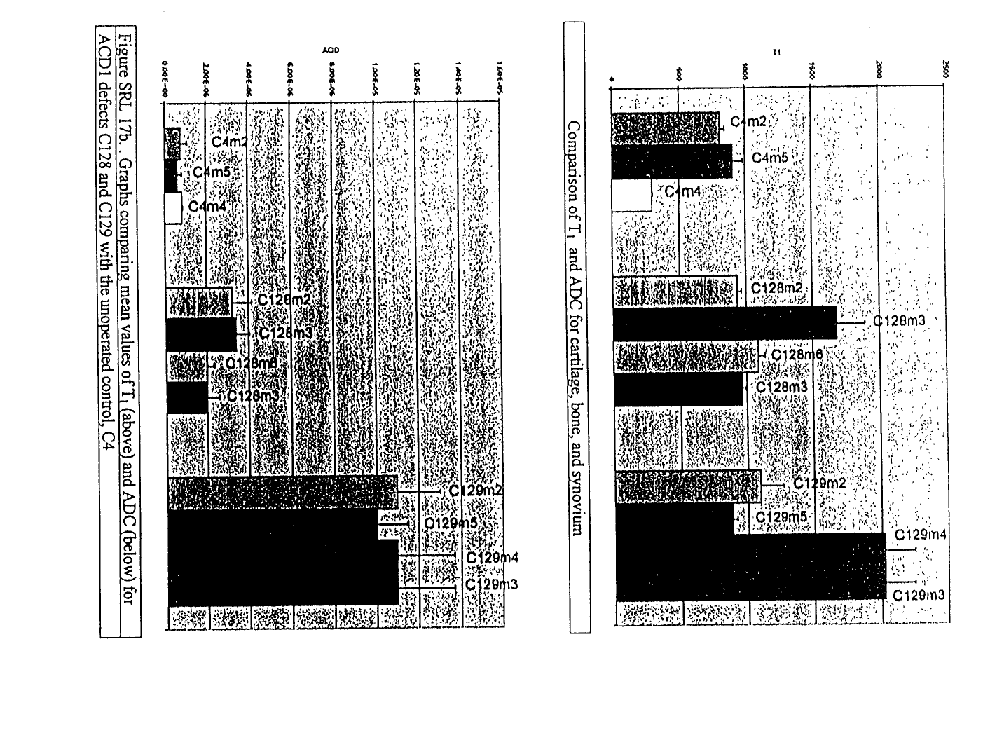

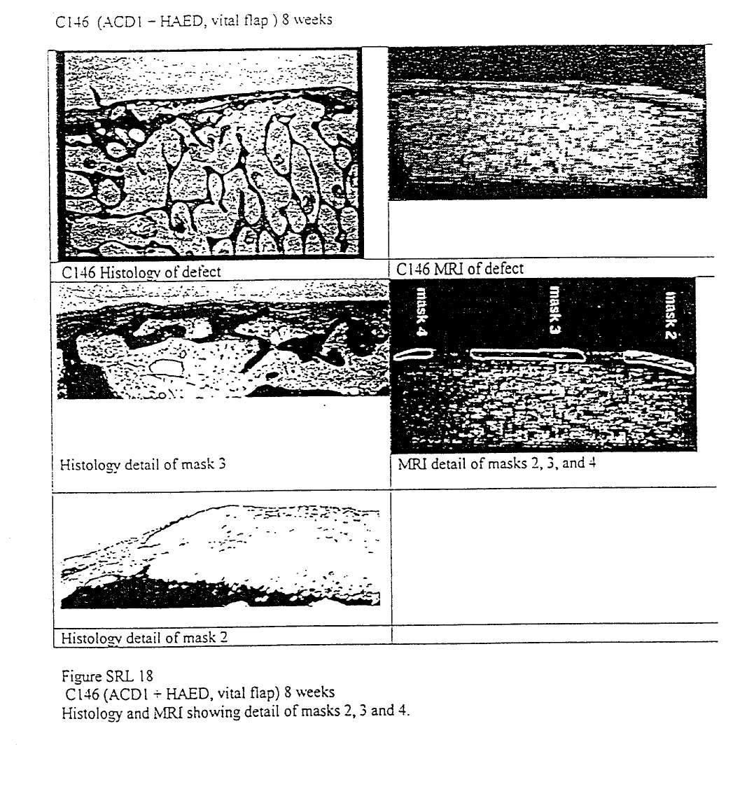

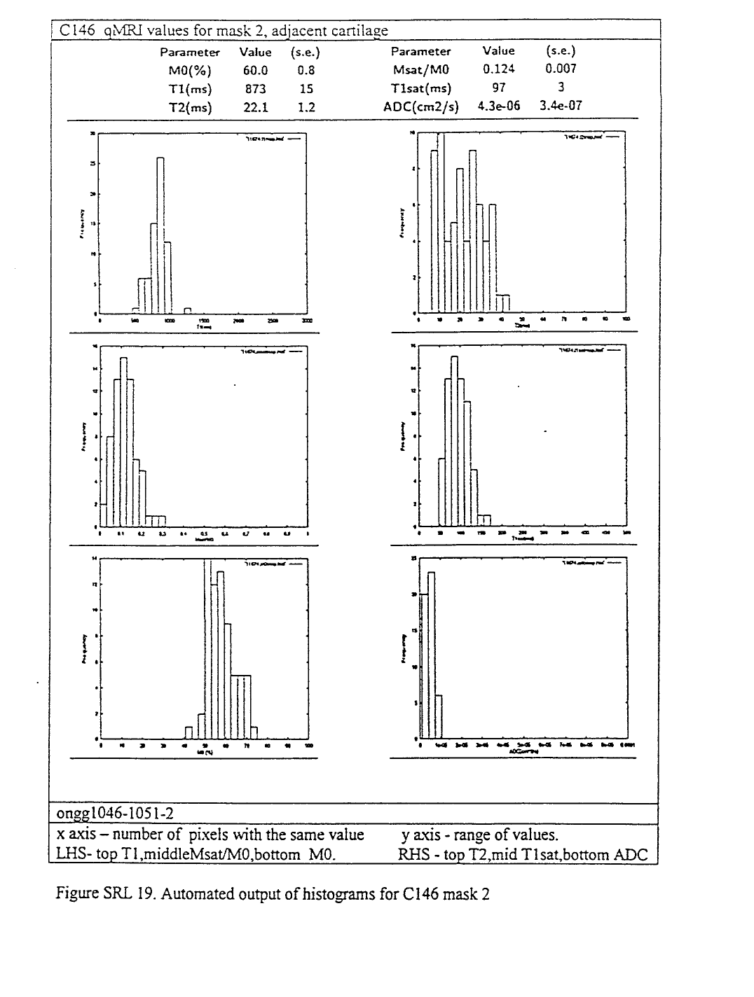

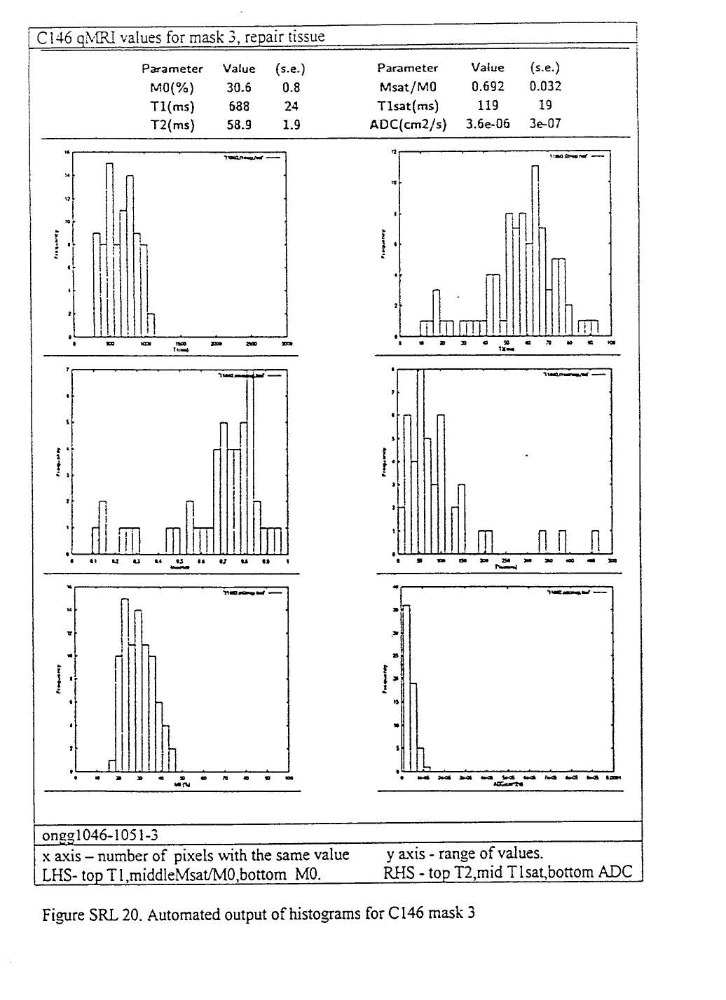

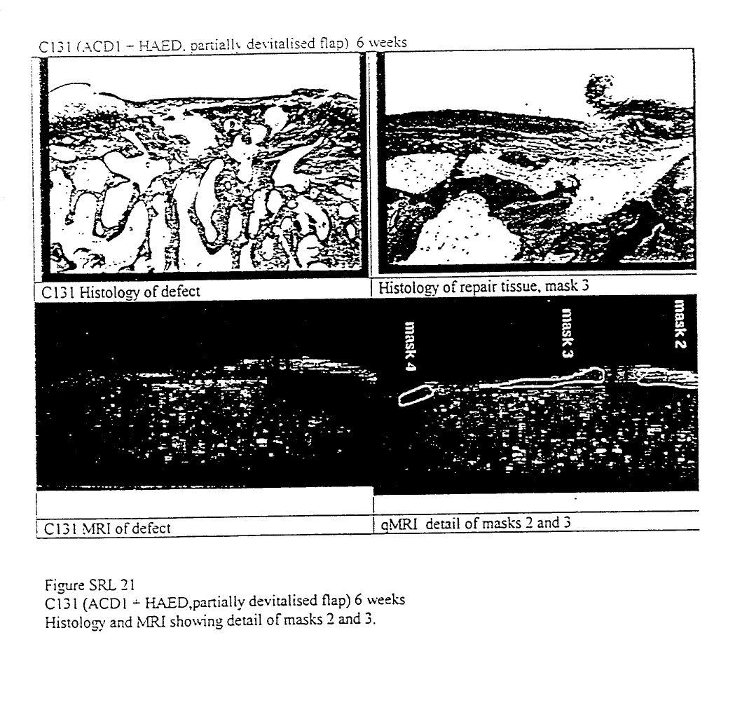

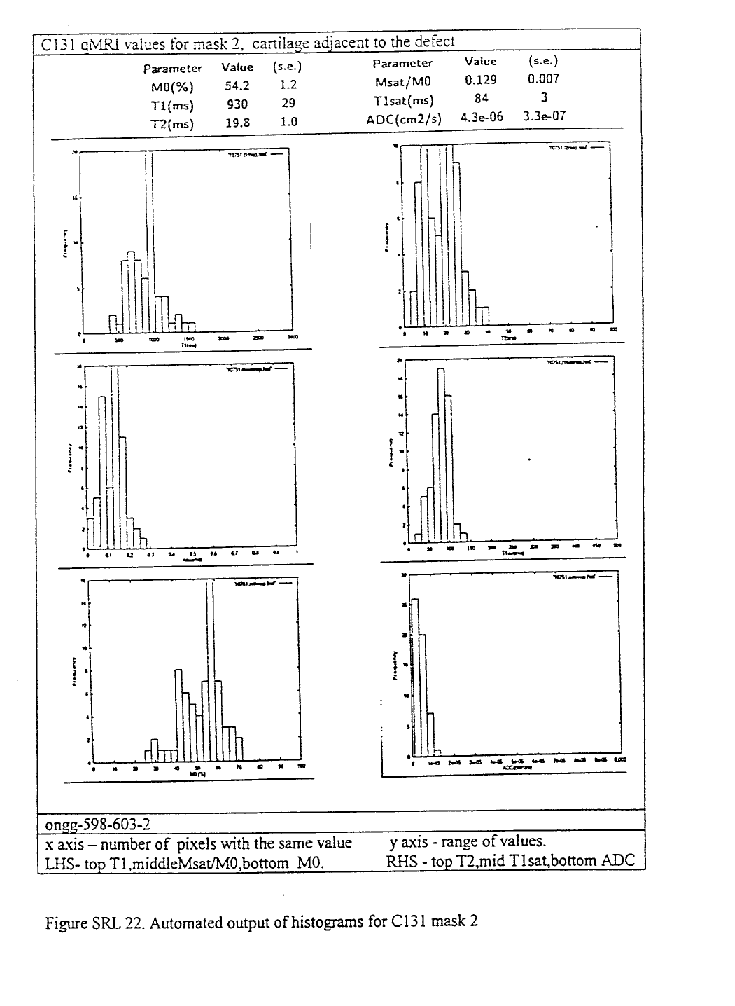

The cartilage defect models are extremely variable and large differences in the amount of matrix retained and degree of transformation were seen for equivalent samples within the same group. C146 (FIGS. 18-20) was mostly empty at 8 weeks but had some repair material within the defect that stained poorly with metachromatic dye and had MR characteristics different to those of cartilage. C131 (FIGS. 21-23) was also partially filled with repair material that stained much darker with metachromatic dye and had MR characteristics within the cartilage range. However, staining with metachromatic dyes is not consistent between samples or even from section to section and it is probably not appropriate to classify the repair tissue on this basis.

Virtual ACD1+PEG Triacrylate Matrix

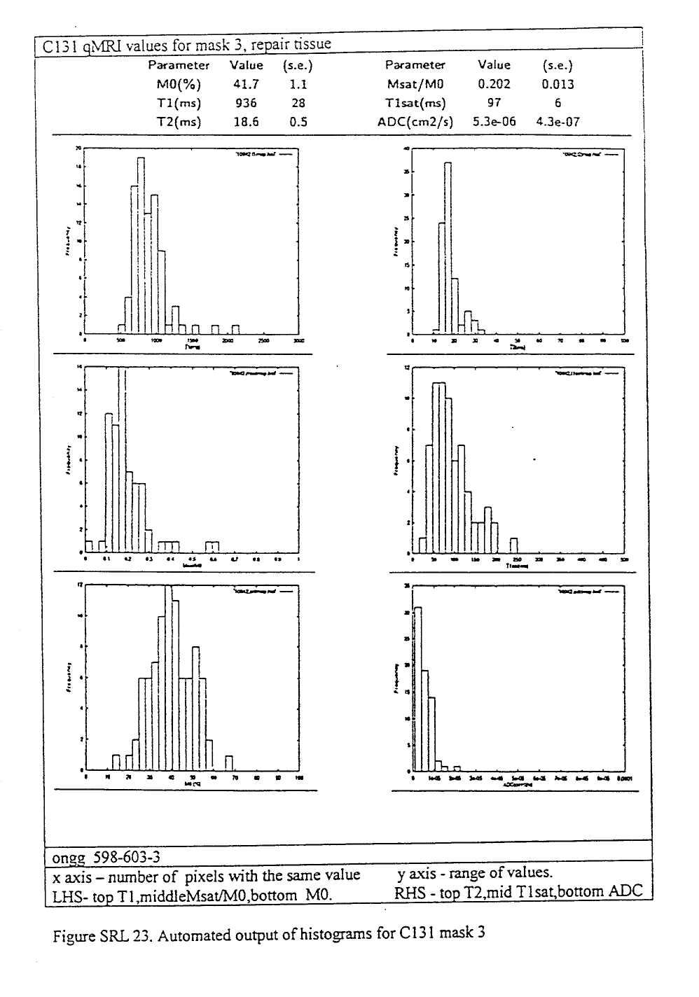

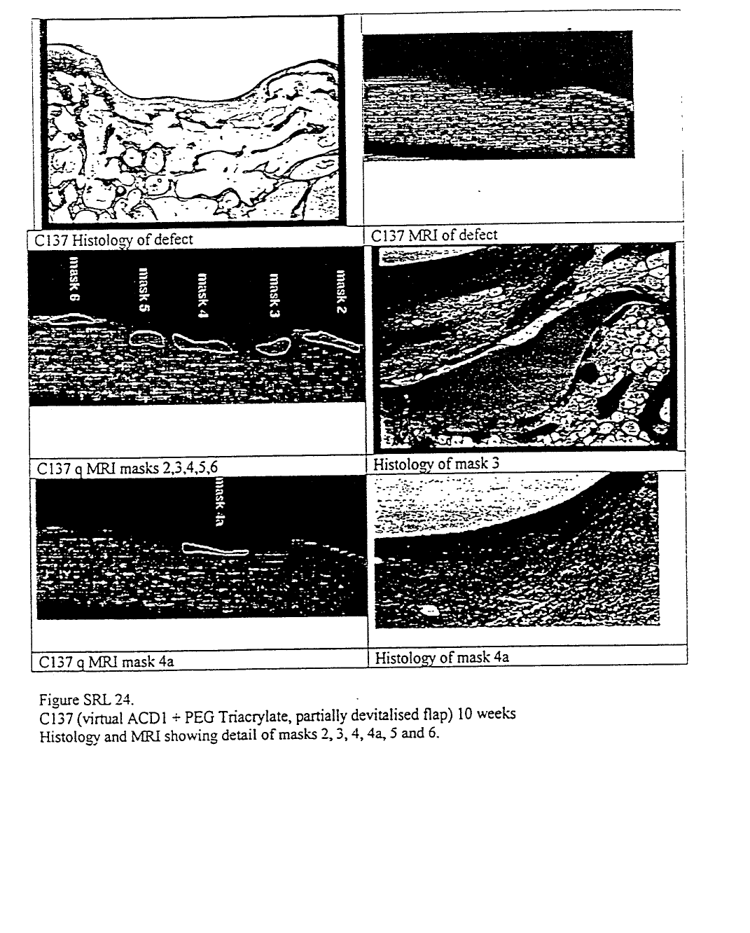

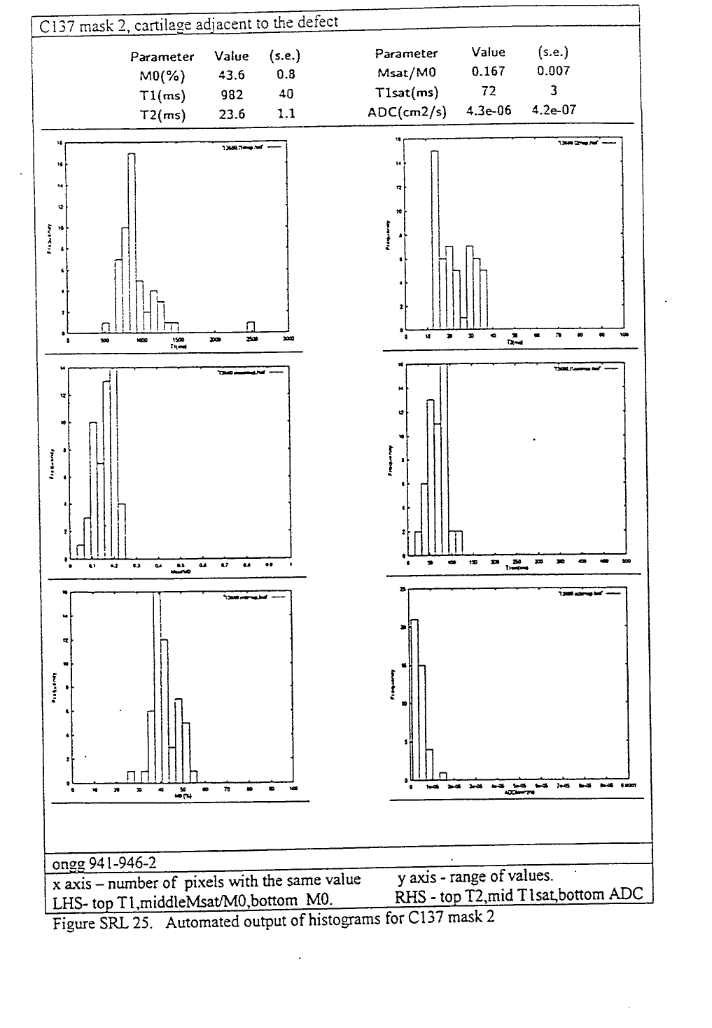

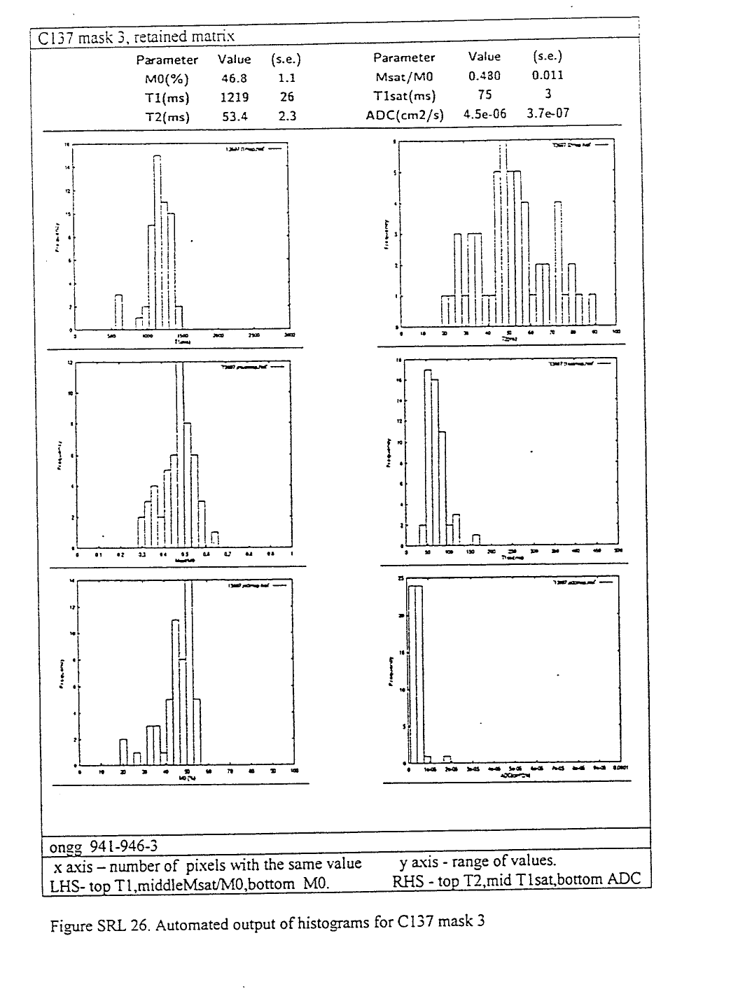

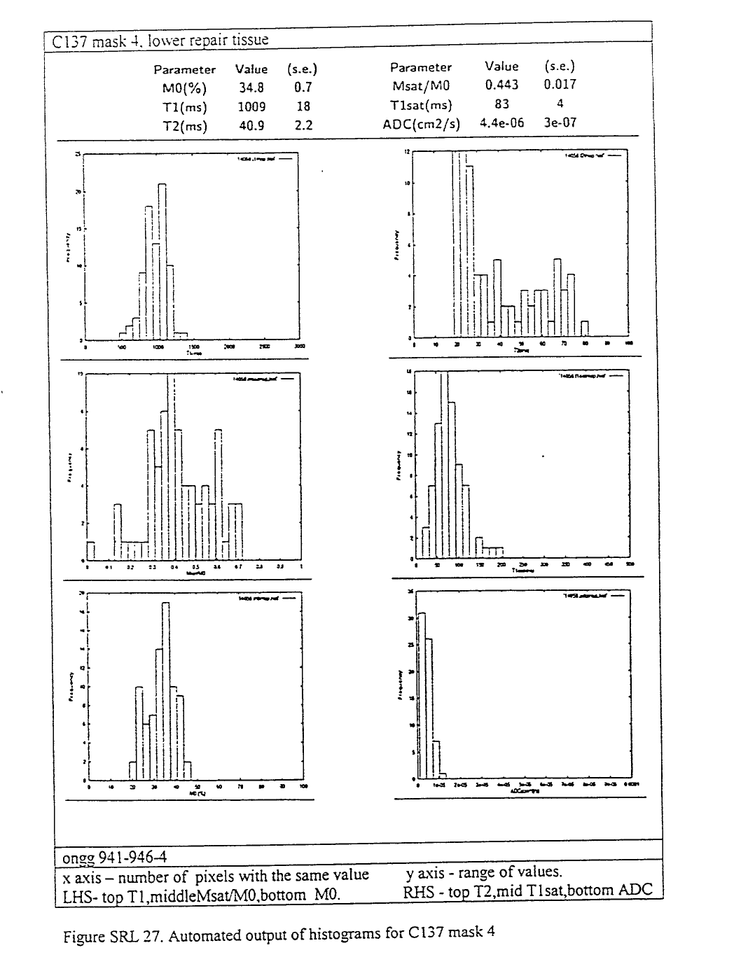

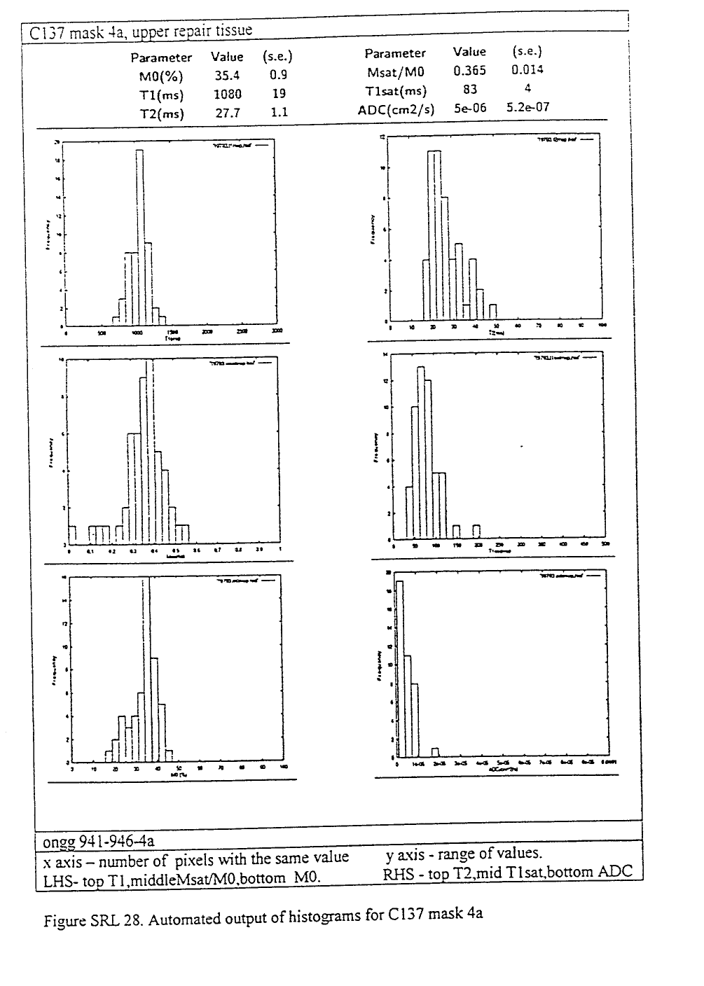

FIGS. 24-29 are views of C137 at 10 weeks. This was a virtual ACD1 defect implanted with PEG Triacrylate to demonstrate that unresorbed matrix retained within the defect is clearly identifiable in the MR image even after several weeks in vivo.

The MR characteristics of repair tissue in this defect were not within the cartilage range.

Spontaneous Repair in ACD3 Defects

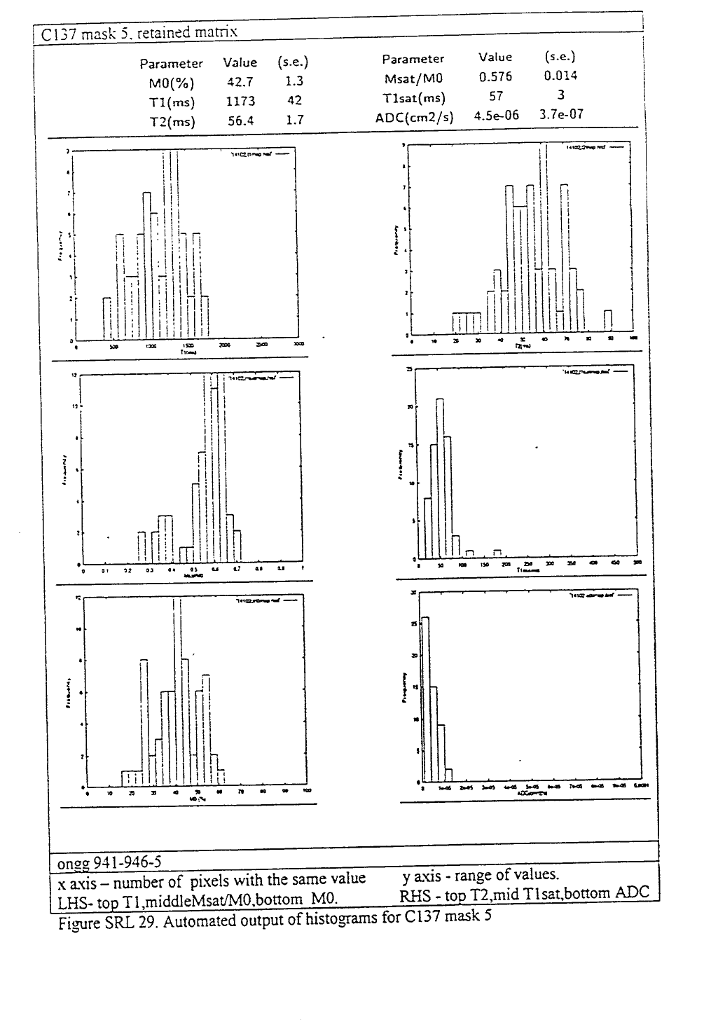

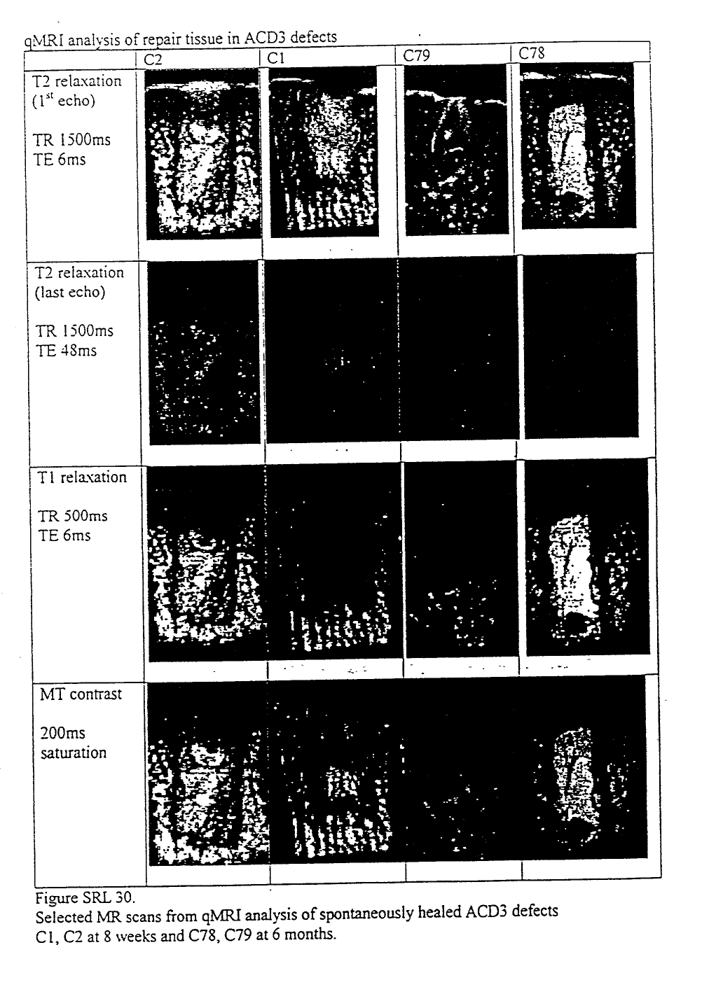

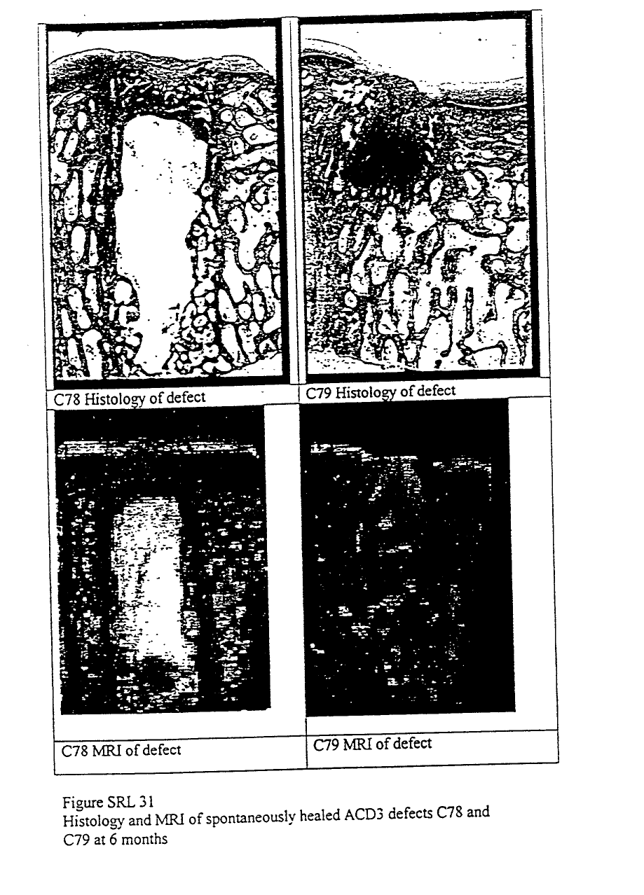

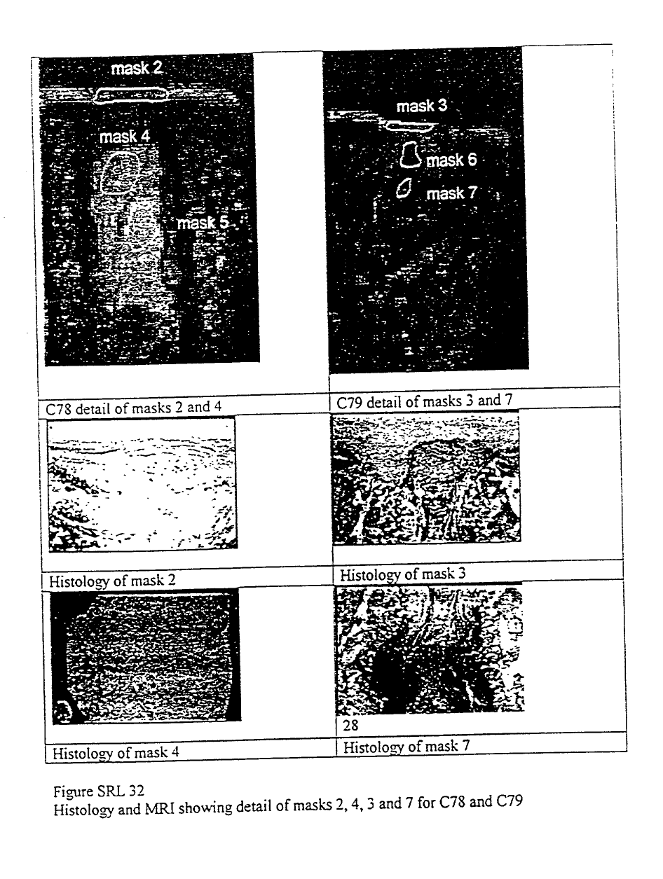

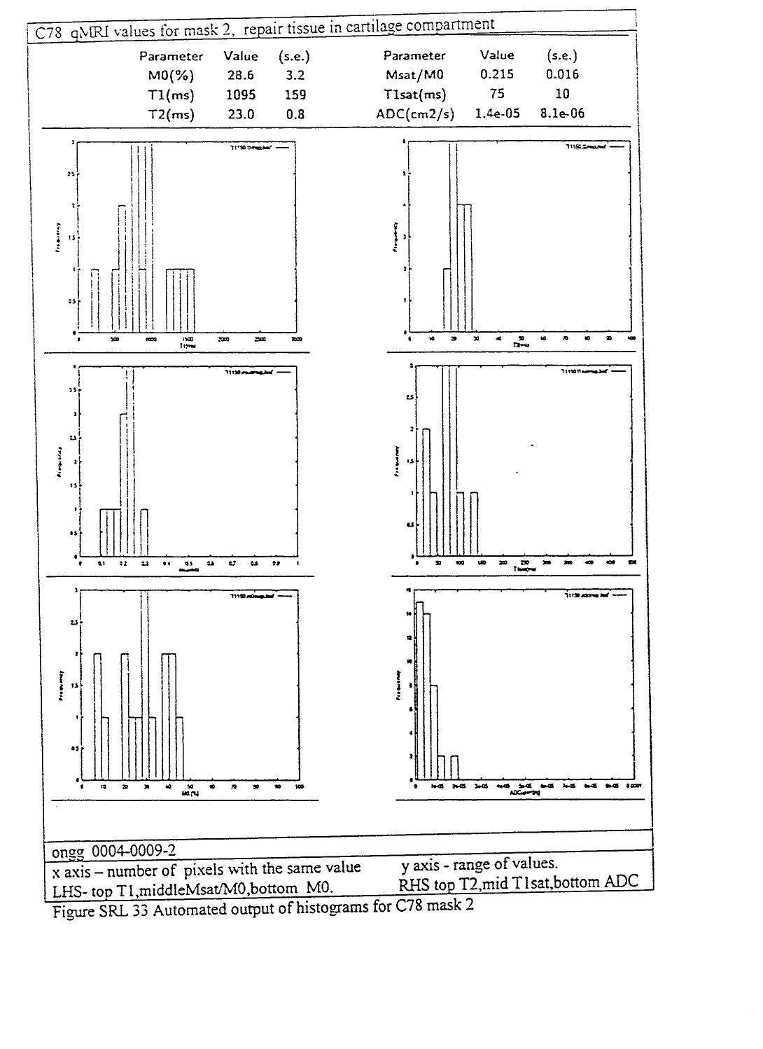

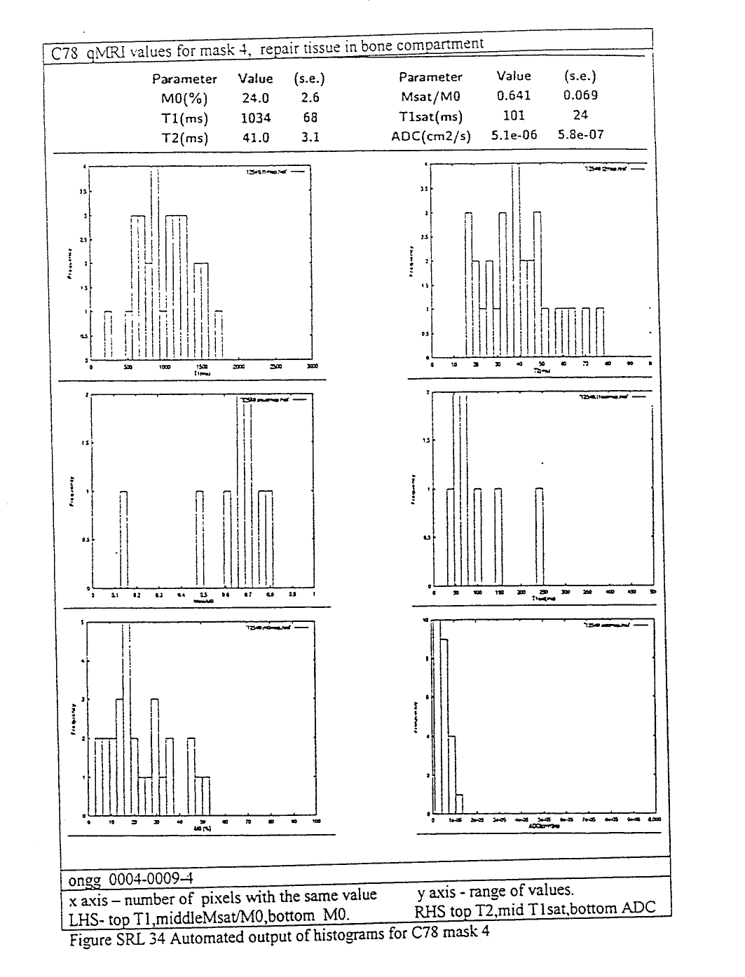

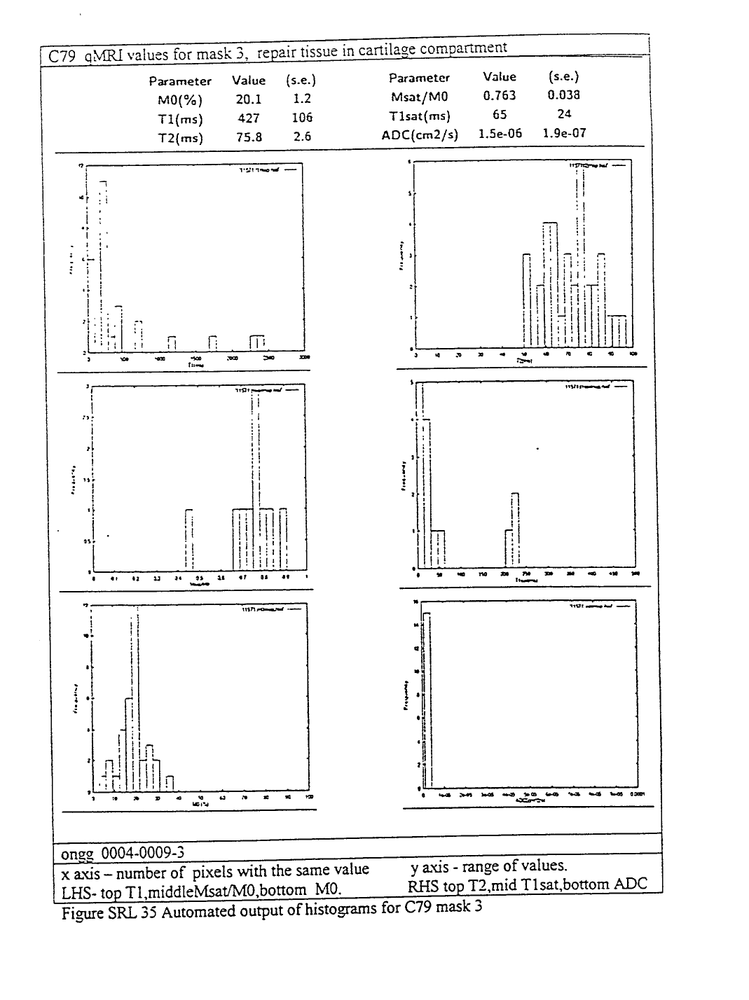

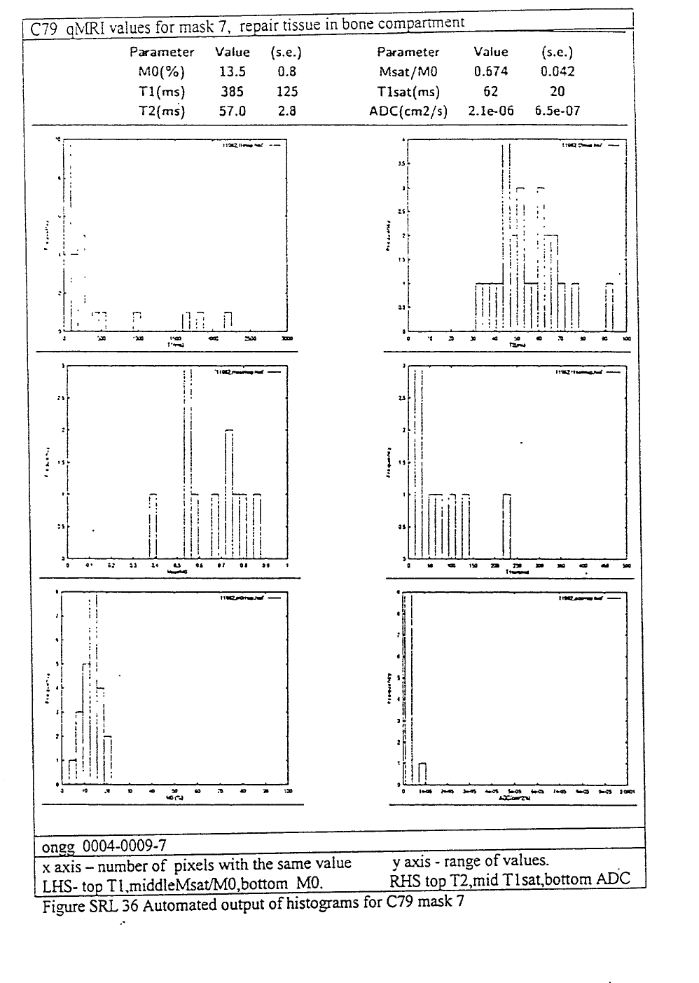

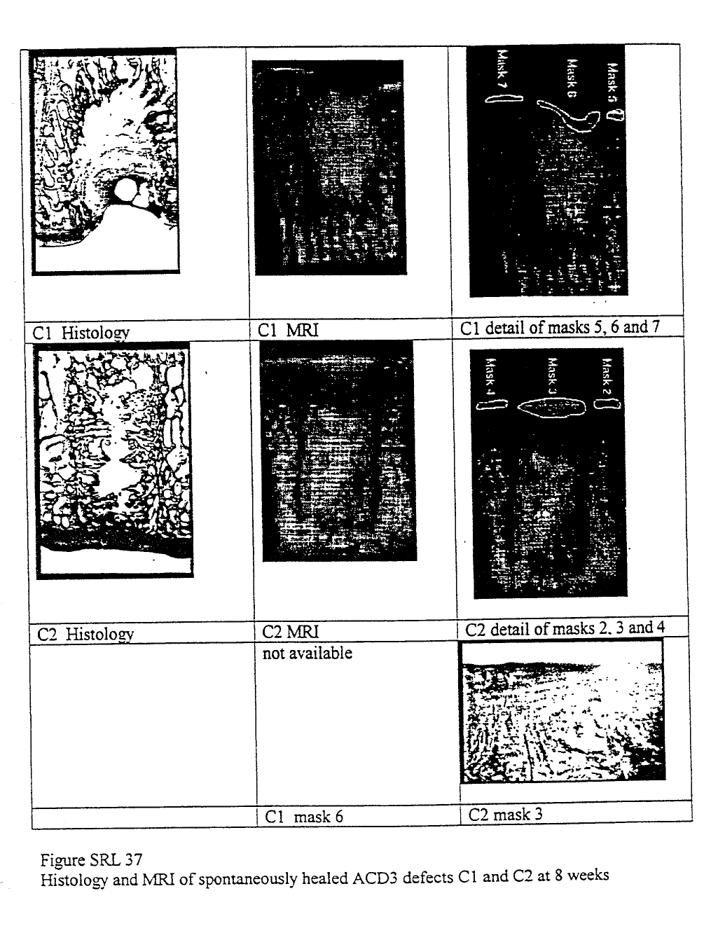

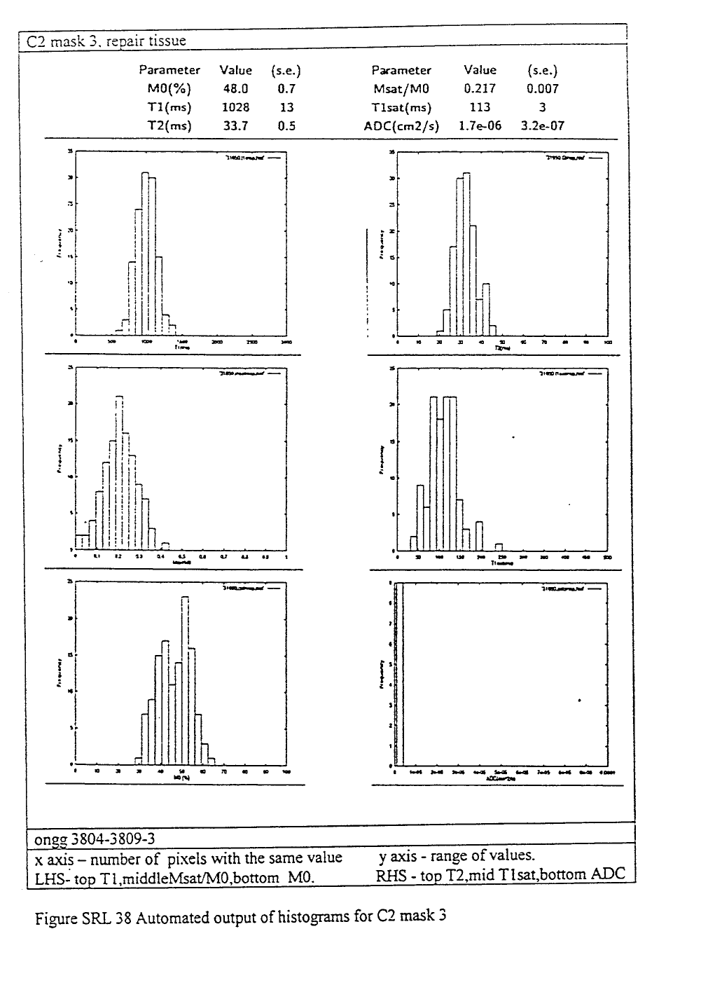

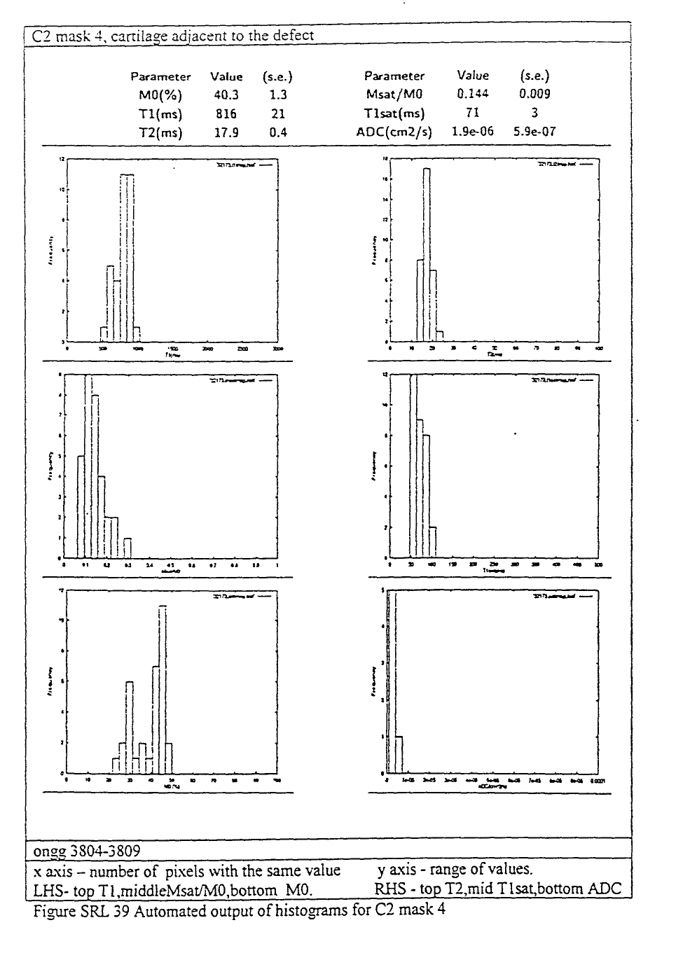

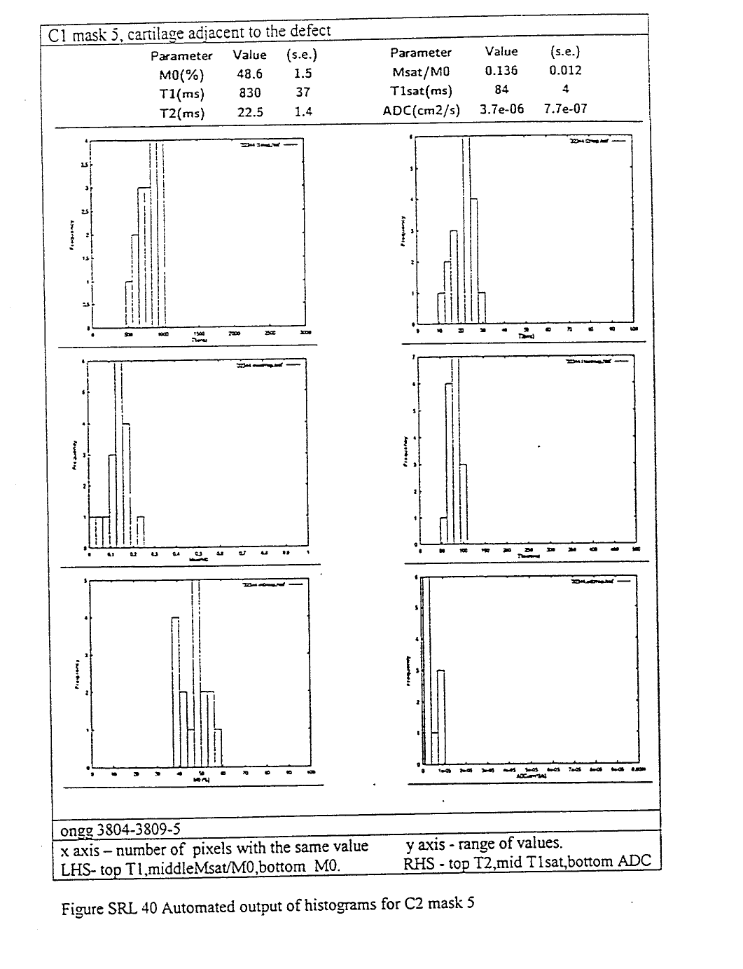

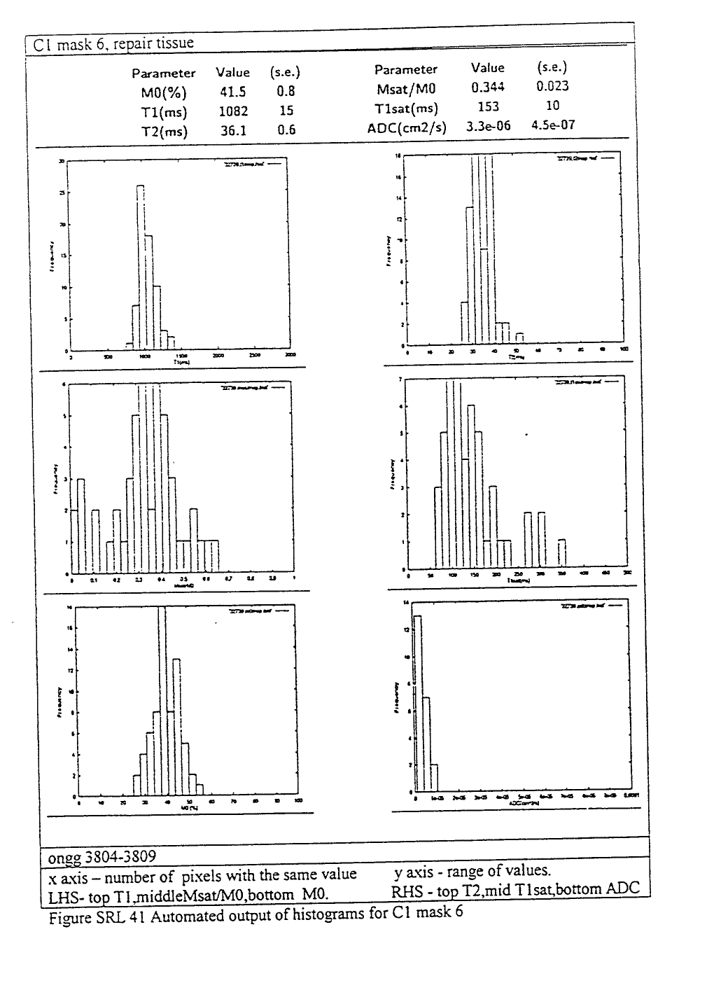

The ACD3 model is also extremely variable. FIG. 30 shows a range of scans from the qMRI analysis for four ACD3 defects that were left to heal spontaneously for 8 or 24 weeks. Large differences in the effectiveness of regenerating both bone and cartilage are clearly visible on the MR images. The MR characteristics (FIGS. 31-36) of repair tissue in the cartilage compartment of C78 are within the cartilage range while those of C79 are not. The mean values for MR parameters (FIGS. 37-42)) of repair tissue in the cartilage compartment of C1 and C2 are both outside the cartilage range. However, it is clear from the histograms that the region within these masks is very heterogeneous and contains several components, one of which is similar to that of a cartilaginous matrix.

ACD3-HAED Matrix

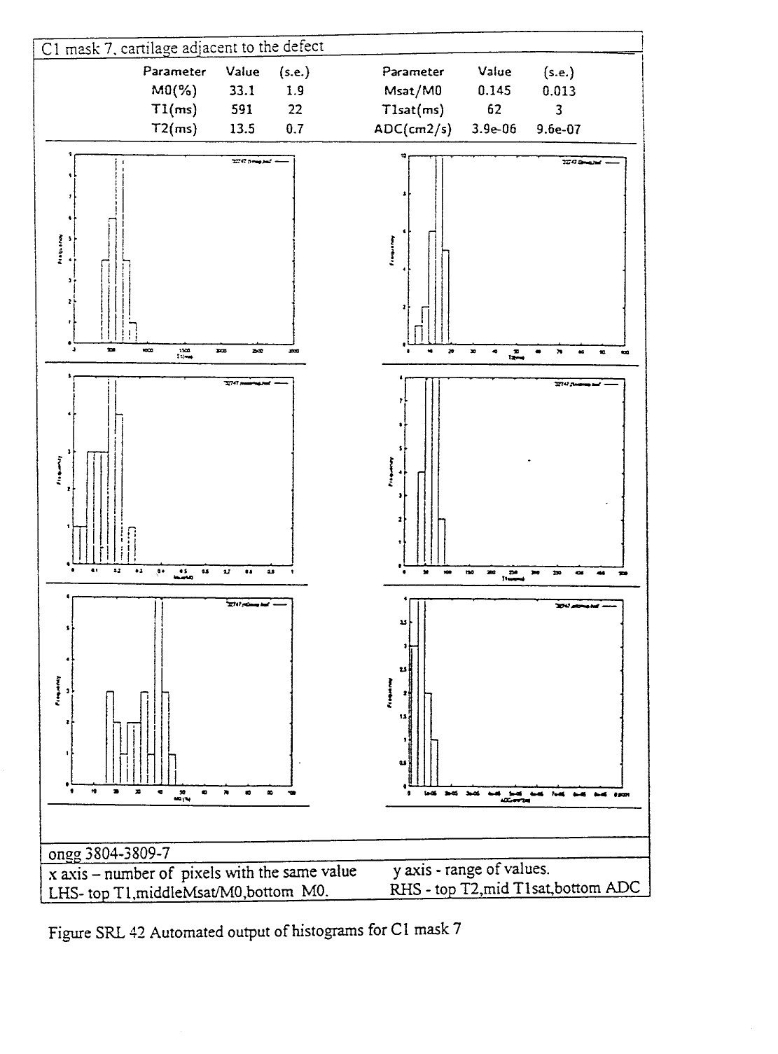

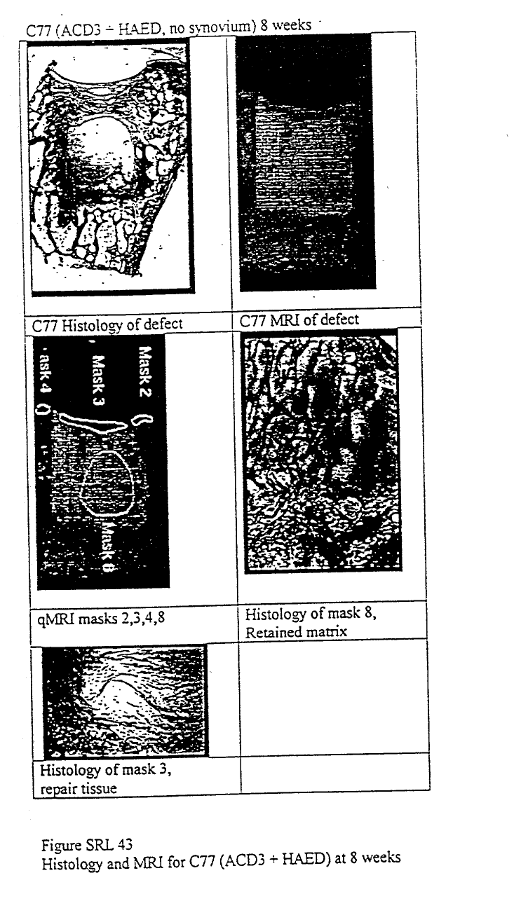

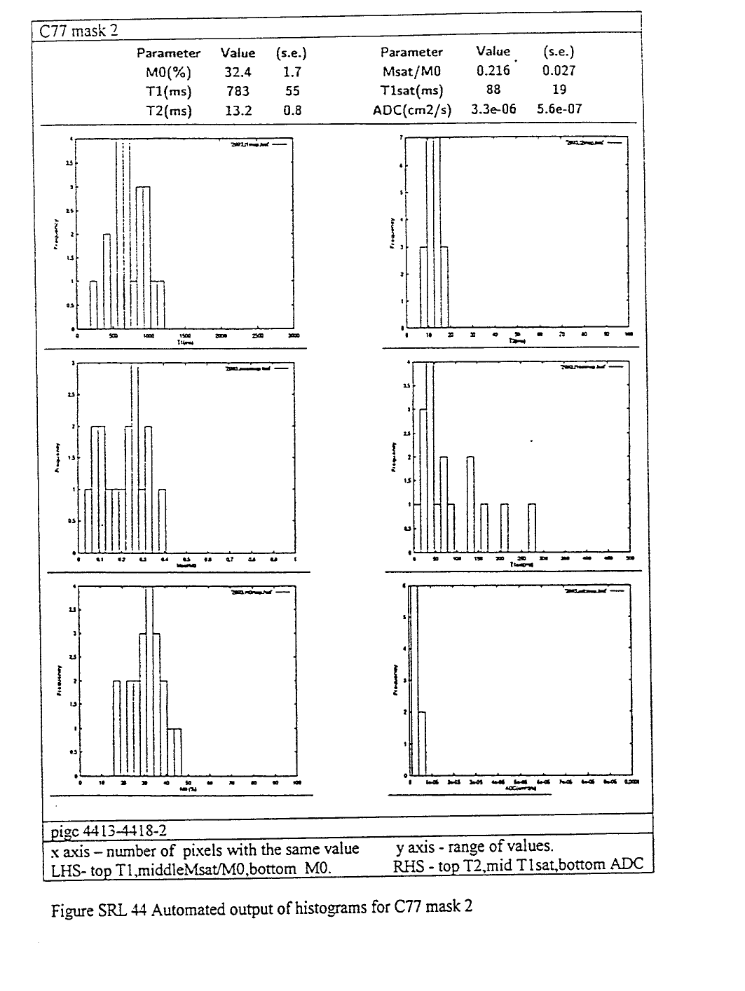

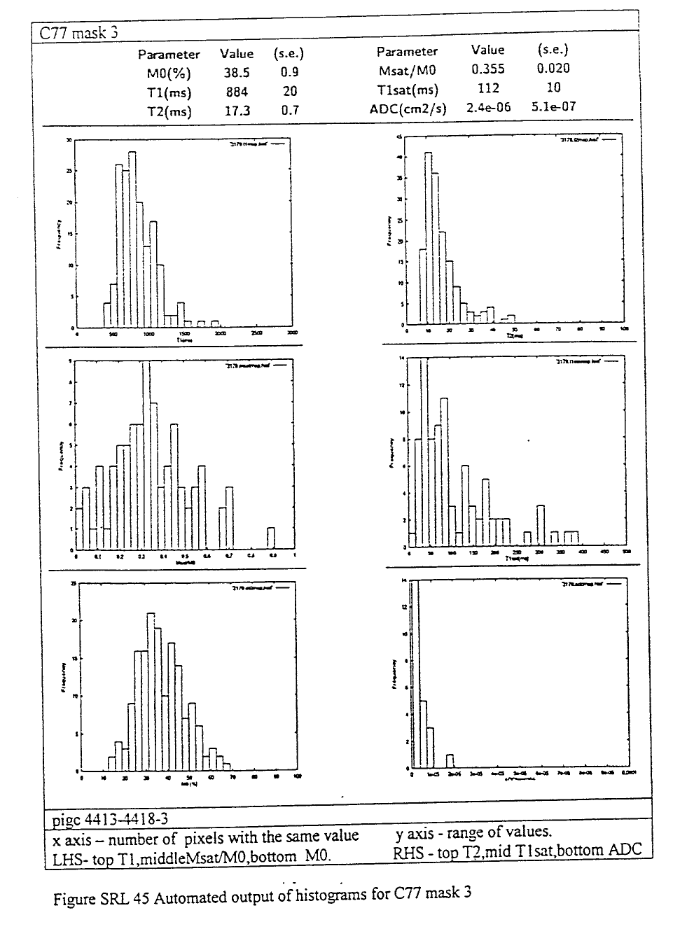

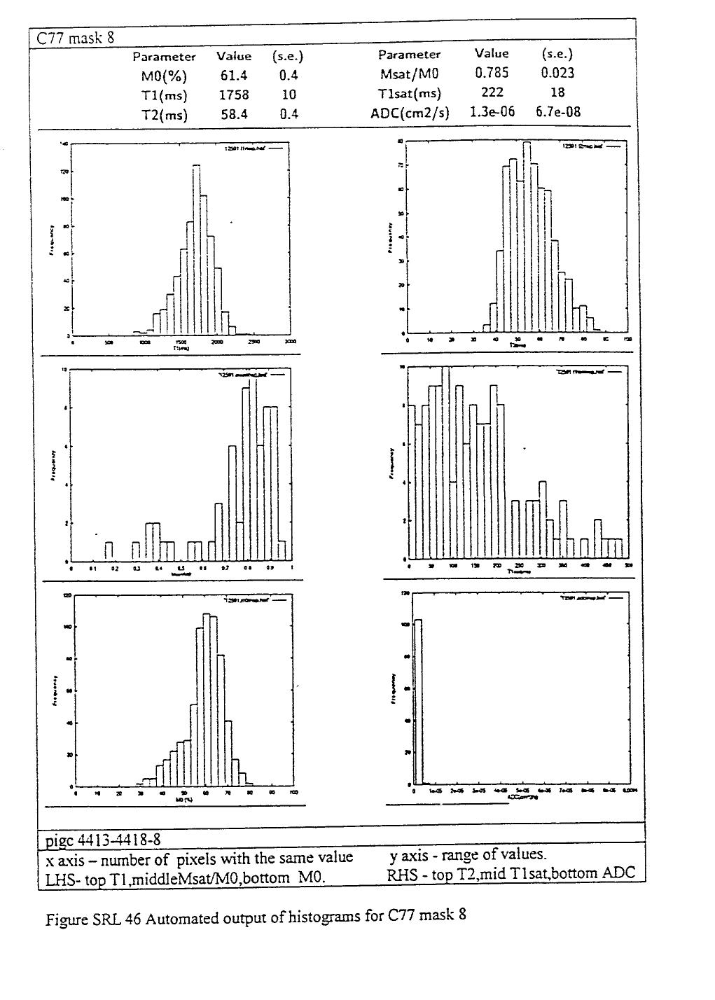

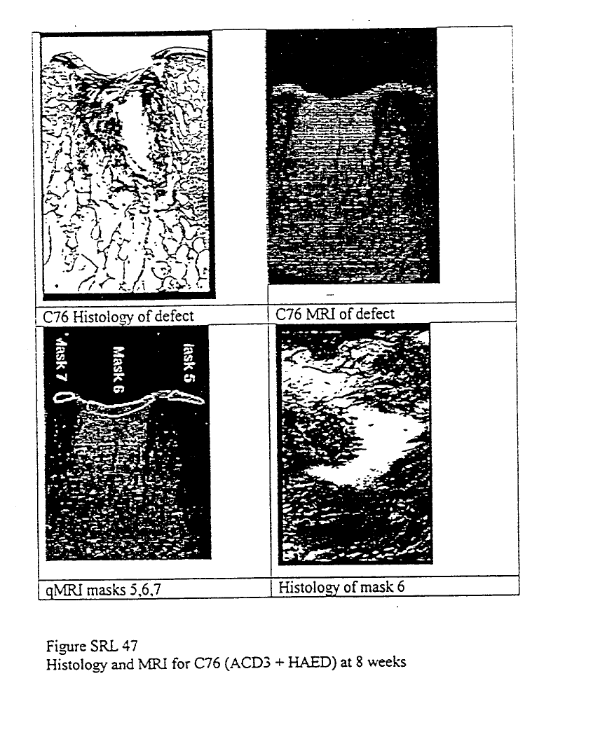

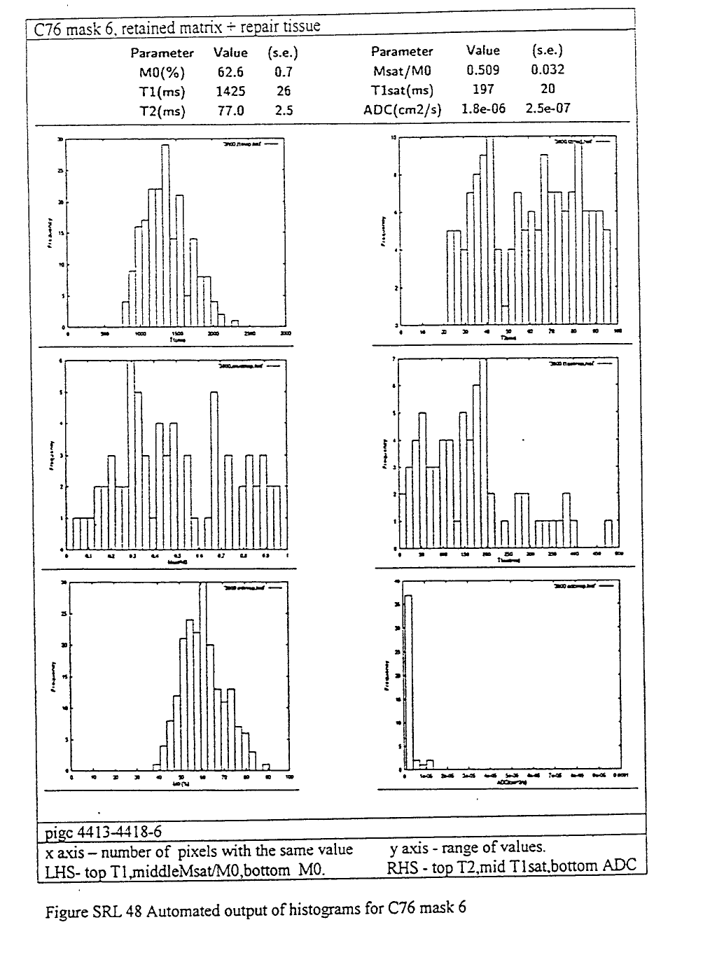

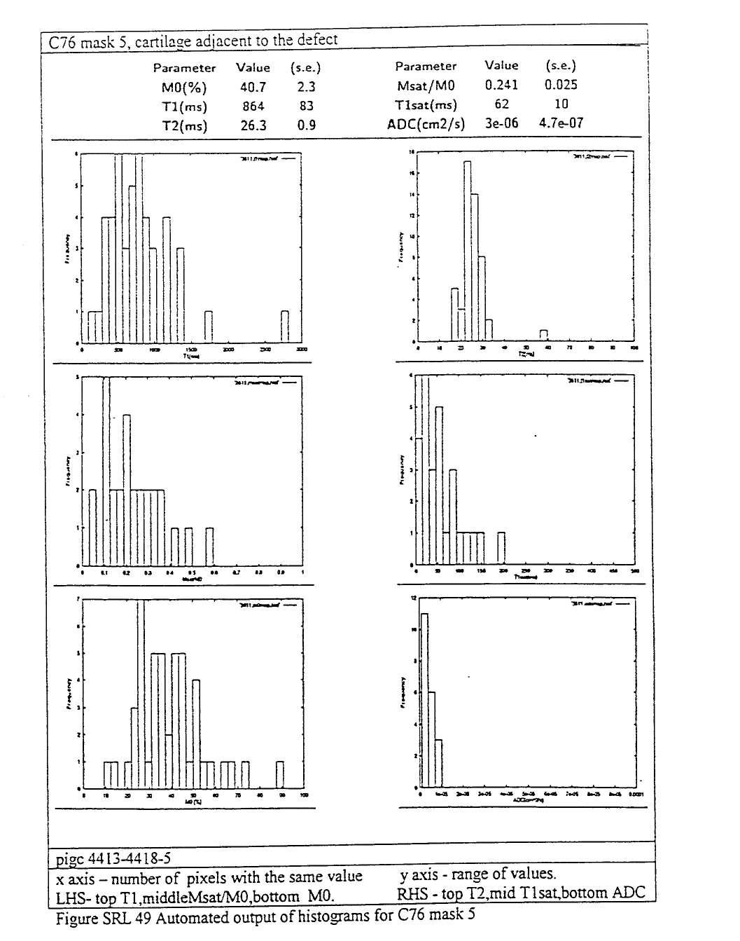

FIGS. 43-49 are views of C76 and C77 at 8 weeks; ACD3 defects filled with HAED. A considerable amount of this matrix is retained in the defect at this time that is easily distinguishable from cartilage or cartilage-like matrix. However, despite being slow to resorb, it is clear (FIG. 47, mask 6) that this matrix is readily infiltrated by cells that induce appropriate repair and both defects contained a component within the masks of the repair tissue that have MR characteristics within the cartilage range. This data is summarized in the graphs in FIGS. 50a and b.

Scoring Repair from 3D Data Sets

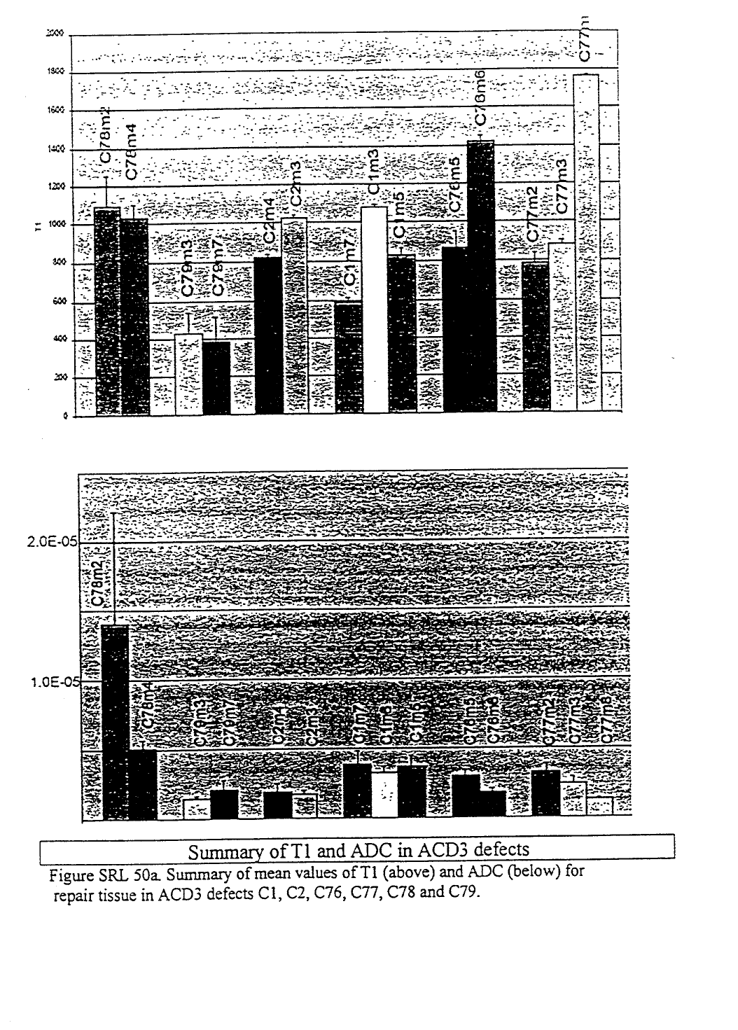

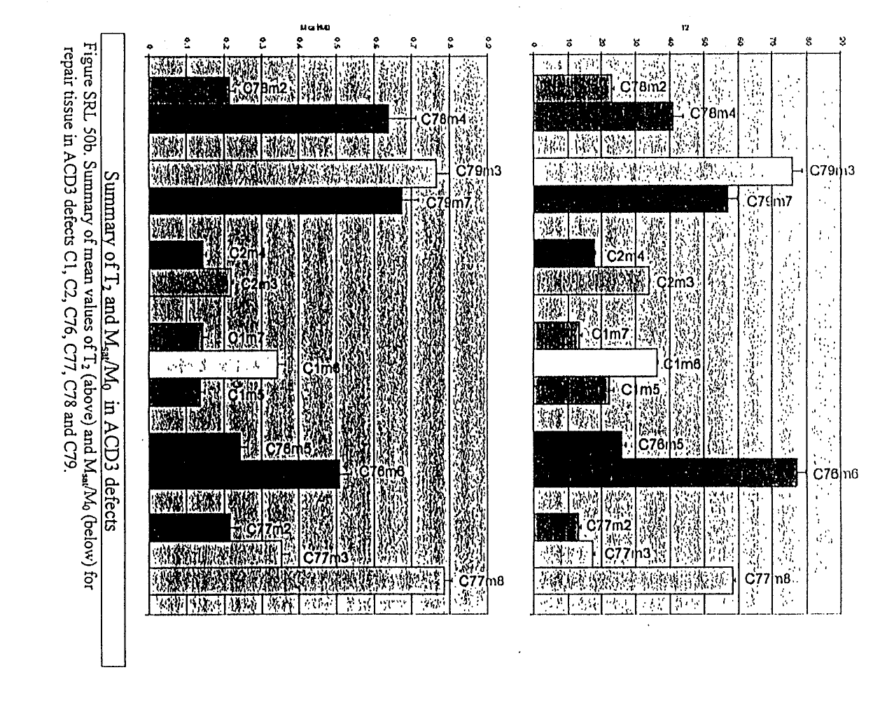

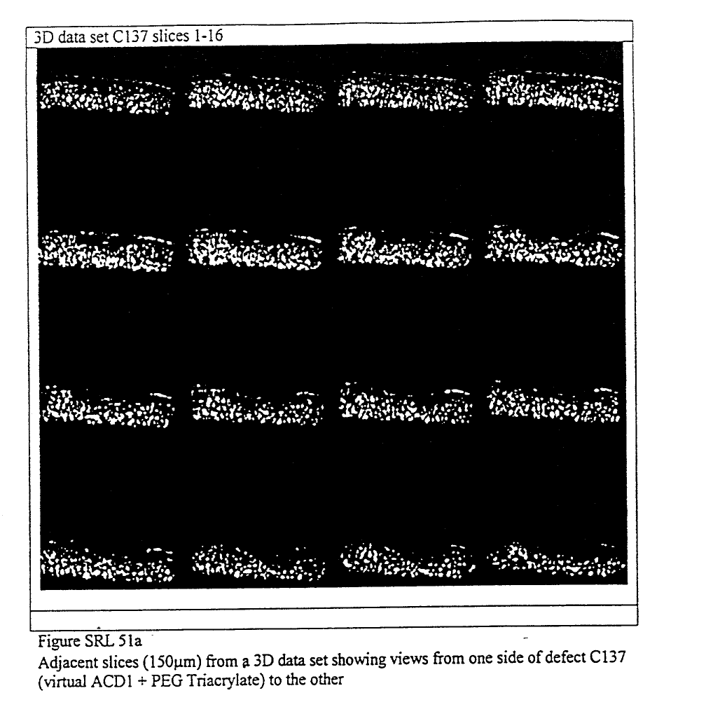

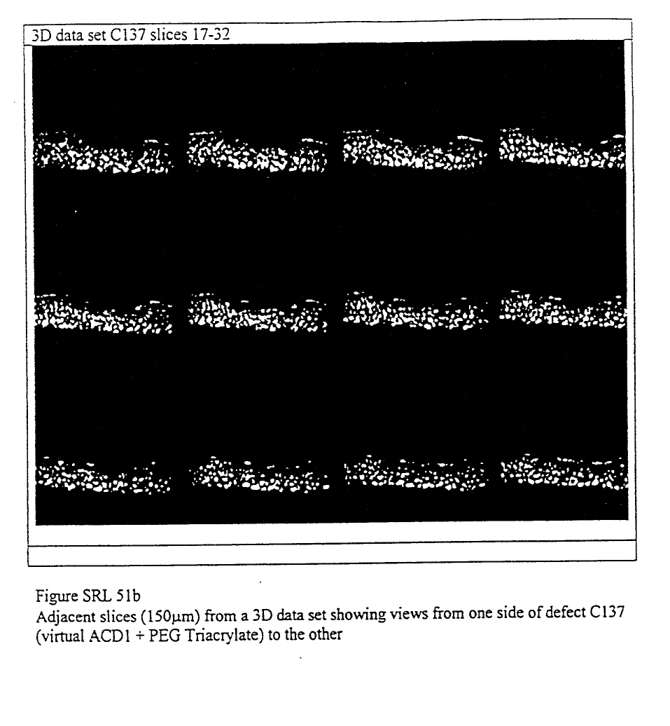

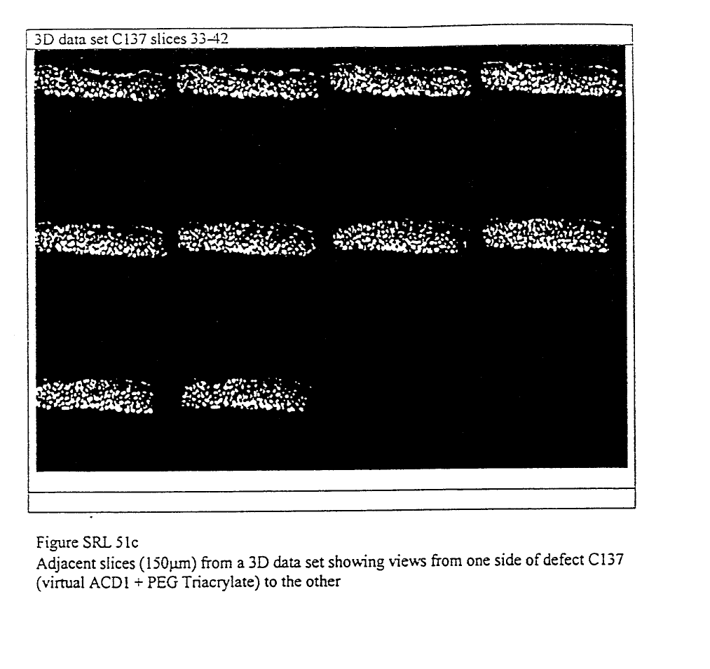

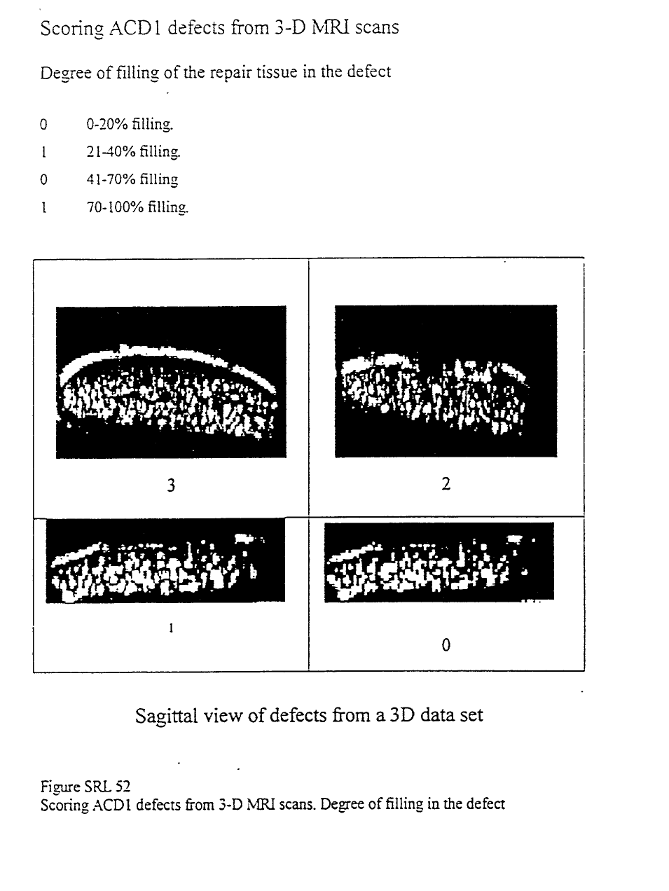

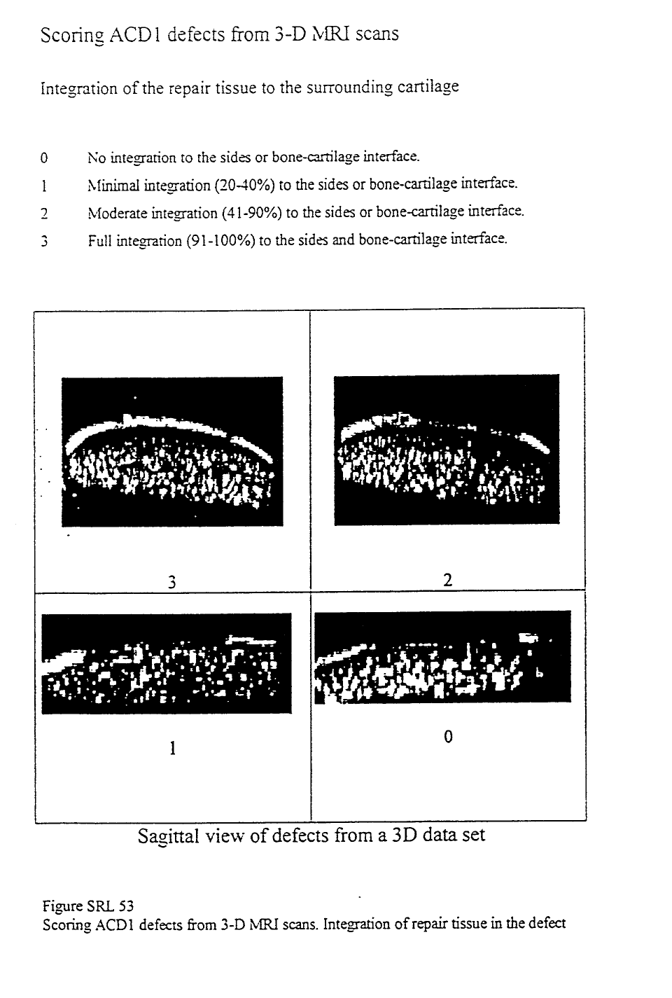

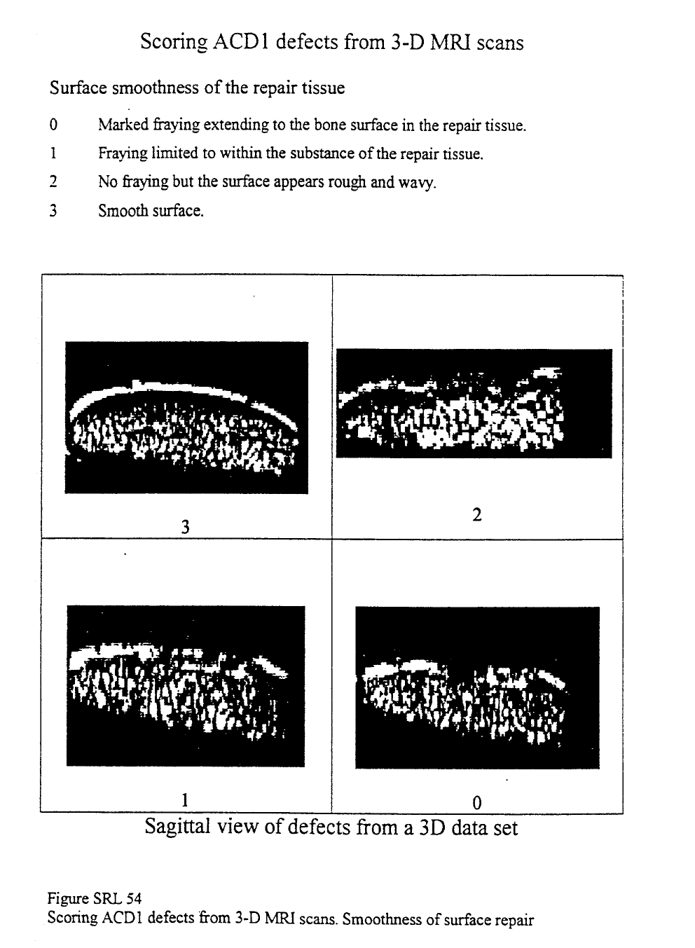

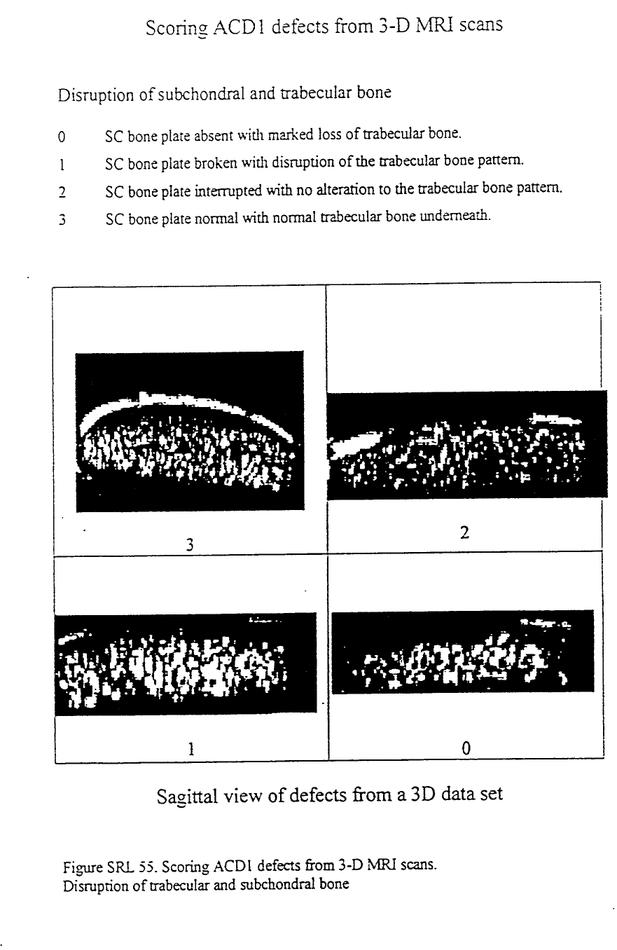

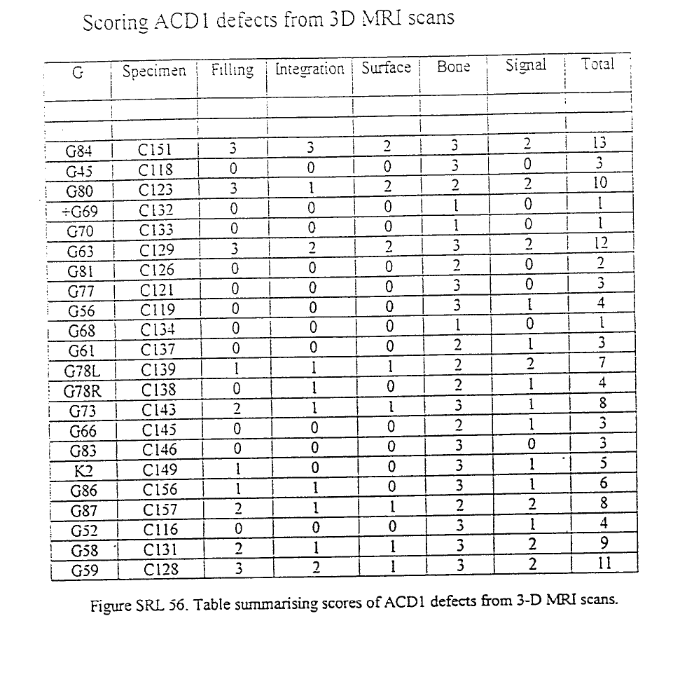

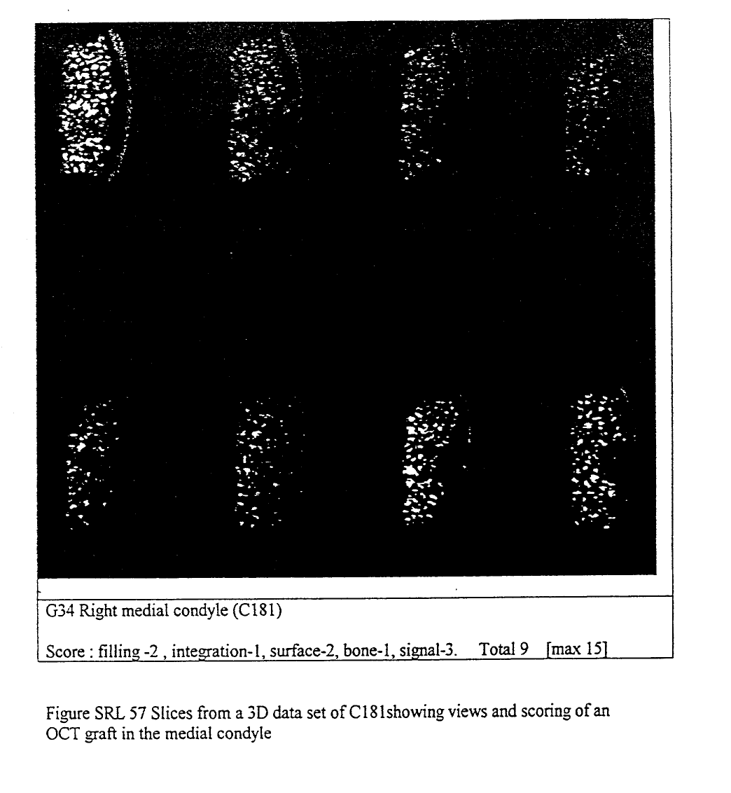

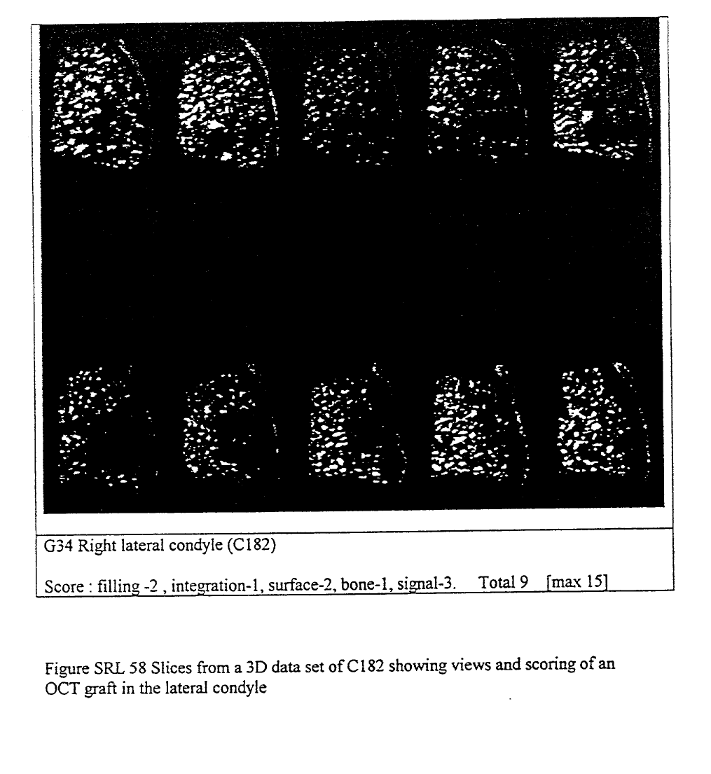

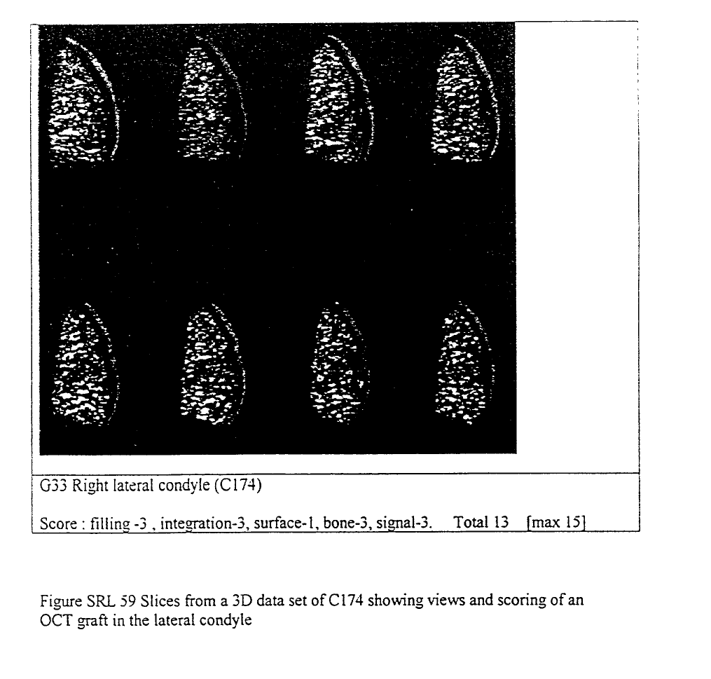

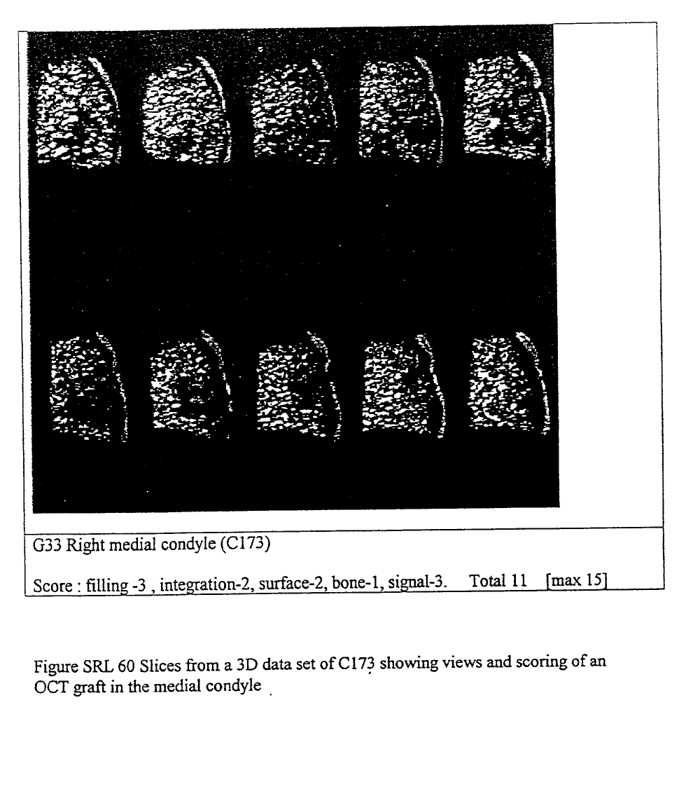

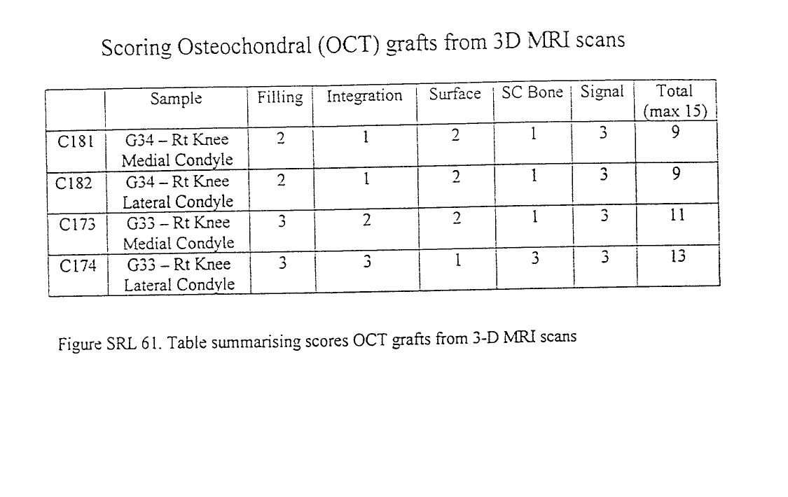

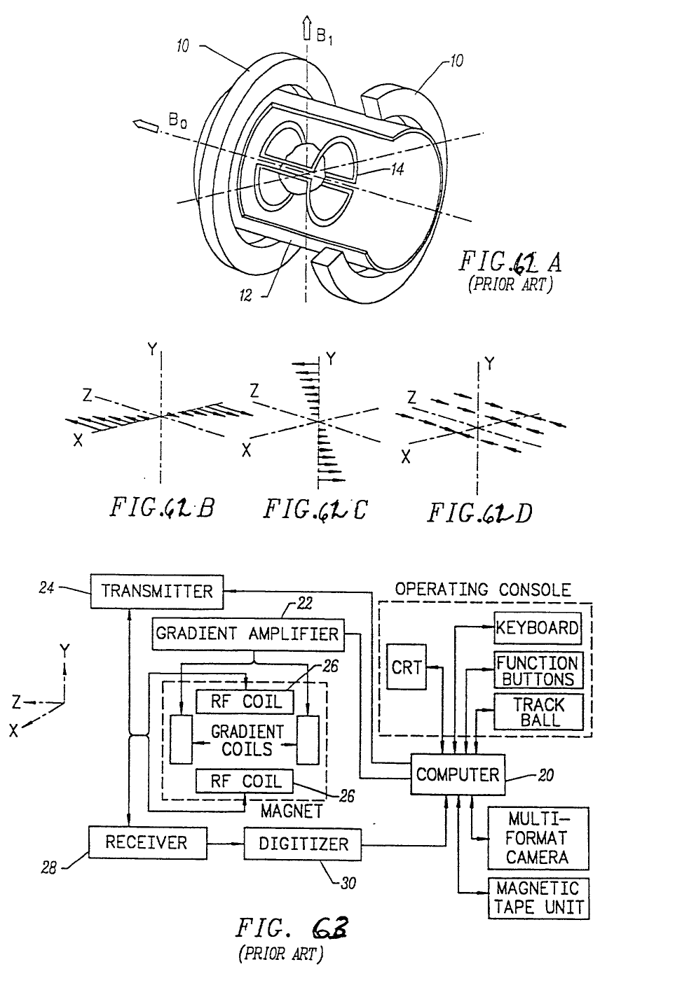

FIGS. 51a-c show adjacent views in one plane from a 3D data set of defect C137. One method of scoring the extent of infilling, integration and bone changes from such data sets are described in FIGS. 52-55. Other schemes are also possible and can be tailored to represent the scoring of features of interest in the same way that histology scoring was used in the present study. The defect is viewed in multiple planes as adjacent sections which are 150 μm apart in these experimental defects but would be 600 μm apart in scans of the human knee. A single composite score per defect is then recorded. Scores recorded to date for ACD1 defects are summarized in the Table in FIG. 56. Similar views and a summary of scores are shown in FIGS. 57-61 for osteochondral grafts in the condyle.

Conclusion

The results from both scoring of 3D data sets and quantitation of MR parameters indicate that MRI can provide an objective means of monitoring the outcome of cartilage repair within the knee of patients in vivo.

The T2 relaxation rate and Magnetization ratio are the most reproducible parameters to obtain and give the most sensitivity with respect to differences between cartilage, fibrous connective tissue and matrix It is clear that recording and tabulating the mean value of the those MR parameters for a given mask representing different regions of repair tissue is not the most informative means of assessing the repair process. For the present application, it is more appropriate to assign a range of values for the three groups and tabulate what proportion of pixels within the repair tissue fall into each category. Those three groups can then be color-coded and a spatial map of where those pixels are located can be printed to provide spatial information of where repair is most successful.

What is claimed is:

1. A method for analyzing tissue based on quantized magnetic resonance data comprising the steps of
a) selecting at least one magnetic resonance parameter to characterize a body part, organ or tissue,
b) selecting a suitable pulse sequence to quantify that selected magnetic resonance parameter,
c) using the selected pulse sequence to acquire multiple sets of magnetic resonance signals from the body part, organ or tissue at an unchanged position relative to the measurement acquisition system,
d) quantifying the magnetic resonance imaging parameters on a pixel by pixel basis,
e) determining biological properties of interest of a body part, organ or tissue structure by biological means including histological, biochemical, histochemical, and biomechanical,
f) correlating quantitative ranges of the selected magnetic resonance parameters with selected biological properties of interest of a body party, organ or tissue.

2. The method as defined by claim 1 wherein in step a) the magnetic resonance parameter is selected from longitudinal relaxation time (T₁), transverse relaxation time (T₂), magnetization transfer (MT), and magnetization ratio (MR).

3. The method as defined by claim 2 wherein the tissue is cartilage.

4. The method as defined by claim 3 and further including the step of:
f) creating an image of the tissue based on representation of sets of one or more quantitative magnetic resonance parameters.

5. The method as defined by claim 1 and further including the step of:
f) creating an image based on representation of sets of one or more quantitative magnetic resonance parameters.

6. A method for analyzing tissue based on quantized magnetic resonance data comprising the steps of
a) acquiring magnetic resonance signals from the tissue,
b) determining at least one magnetic resonance quality of tissue in each pixel,
c) quantizing the magnetic resonance signals pixel by pixel within the tissue, and
d) correlating the determined magnetic resonance quality with known magnetic resonance qualities of tissue.

7. The method as defined by claim 6 wherein in step c) the magnetic resonance quality is selected from longitudinal relaxation time (T₁), transverse relaxation time (T₂), magnetization transfer (MT), and magnetization ratio (MR).

8. The method as defined by claim 7 wherein the tissue is cartilage.

9. The method as defined by claim 8 and further including the step of:
d) creating an image of the tissue based on the determined magnetic resonance quality.

10. The method as defined by claim 6 and further including the step of:
d) creating an image of the tissue based on the determined magnetic resonance quality.

11. Magnetic resonance apparatus for use in analyzing a body comprising:
a) means for establishing a magnetic field through the body,
b) means for exciting nuclei spins in the body with an RF signal oriented at an angle with respect to said magnetic field,
c) means for receiving magnetic resonance signals from the excited nuclei representative of said nuclei spins,
d) repeating steps b) and c) to obtain a multiplicity of sets of magnetic resonance signals and determining a magnetic resonance quality from the body, and
e) means for quantizing the magnetic resonance quality pixel by pixel within the body.

12. Apparatus as defined by claim 11 wherein the magnetic resonance quality is T2 relaxation time.

13. Apparatus as defined by claim 12 wherein steps b), c), and d) are pulse echo sequences with varying echo times.

14. Apparatus as defined by claim 11 wherein the magnetic resonance quality is chosen form T1 relaxation time, T2 relaxation time, and magnetic ratio.

15. Apparatus as defined by claim 11 and further including
f) a display for imaging the magnetic resonance qualities pixel by pixel.
